(19) United States
(12) Patent Application Publication
(10) Pub. No.: US 2023/0152314 A1
(43) Pub. Date: May 18, 2023

(54) LIPOCALIN-2 ASSAY FOR INTESTINAL ADAPTATION

(71) Applicant: THE JOHNS HOPKINS UNIVERSITY, Baltimore, MD (US)

(72) Inventor: Samuel M. Alaish, Baltimore, MD (US)

(21) Appl. No.: 17/995,882

(22) PCT Filed: Apr. 12, 2021
(86) PCT No.: PCT/US2021/026806

§ 371 Date: Oct. 10, 2022

Related U.S. Application Data
(60) Provisional application No. 63/008,354, filed on Apr. 10, 2020.

(51) Int. Cl. G01N 33/569; A61K 38/20; A61P 1/00

(52) U.S. Cl. CPC G01N 33/56966; A61K 38/20; A61P 1/00

(57) ABSTRACT

The present invention relates to the field of short bowel syndrome. More specifically, the present invention provides methods and compositions useful for assaying for intestinal adaptation. In a specific embodiment, a method for treating a patient having SBS who is undergoing parenteral nutrition comprises the steps of (a) measuring lipocalin-2 (LCN2) in a sample obtained from the patient; and (b) reducing or eliminating parenteral nutrition if the measured level of LCN2 is below a control level or treating the patient with IL-22, a LCN2 inhibitor and/or an AHR agonist if the measured level of LCN2 is above the control level.

### CROSS-REFERENCE TO RELATED APPLICATIONS

This application claims the benefit of U.S. Provisional Application No. 63/008,354, filed Apr. 10, 2020, which is incorporated herein by reference in its entirety.

### FIELD OF THE INVENTION

The present invention relates to the field of short bowel syndrome. More specifically, the present invention provides methods and compositions useful for assaying for intestinal adaptation.

### INCORPORATION-BY-REFERENCE OF MATERIAL SUBMITTED ELECTRONICALLY

This application contains a sequence listing. It has been submitted electronically via EFS-Web as an ASCII text file entitled “P15751-02_ST25.txt.” The sequence listing is 10,371 bytes in size, and was created on Apr. 9, 2021. It is hereby incorporated by reference in its entirety.

### BACKGROUND OF THE INVENTION

The malabsorptive state known as short bowel syndrome (SBS) has devastating consequences. Most SBS patients are infants, who typically develop SBS following an extensive bowel resection for either a congenital anomaly or a postnatal condition, such as necrotizing enterocolitis.^{1, 2} SBS patients develop intestinal failure and require prolonged parenteral nutrition.¹⁻³ Complications of SBS include cholestasis,¹⁻³ increased intestinal permeability¹ and an intestinal dysbiosis with a decrease in bacterial diversity and an increase in Proteobacteria, which produce lipopolysaccharide (LPS) and are pro-inflammatory in nature.⁴⁻⁶ Intestinal permeability increases with this intestinal dysbiosis,⁷ and these recurrent infections are known to increase liver dysfunction⁸ and reduce bile flow into the intestine which weakens the intestinal barrier.^{9, 10} Current clinical management of SBS patients to derail this vicious cycle is limited to prophylactic antibiotics to combat the dysbiosis and decrease infections;^{1, 2} however, this therapy has low efficacy with many adverse effects, including the emergence of resistant bacteria and fungal overgrowth, culminating in formidable infections. A better understanding of the mechanisms involved in intestinal barrier homeostasis during this period of adaptation is vital to augmenting the adaptive response through new focused molecular therapies.

### SUMMARY OF THE INVENTION

Currently, physicians have limited ability to give prognostic information to short bowel syndrome patients and their families with regard to the duration of parenteral nutrition they may require. SBS affects several thousand children per year as well as an increasing number of adults.

There is currently an assay for citrulline which correlates with the amount of residual intestine the patient has. As described herein, the assay of the present invention correlates to the function of the remaining intestine (i.e., does it behave like adapted intestine or inflamed, diseased gut). More specifically, in certain embodiments, a stool assay measuring lipocalin-2 correlates with intestinal adaptation in a mouse model of short bowel syndrome. This assay could be easily used to screen short bowel syndrome patients for prognosis and potential to wean from parenteral nutrition.

Accordingly, in one aspect, the present invention provides compositions and methods useful for treating a patient having SBS. In a specific embodiment, a method for treating a patient having SBS who is undergoing parenteral nutrition comprises the steps of (a) measuring lipocalin-2 (LCN2) in a sample obtained from the patient; and (b) reducing or eliminating parenteral nutrition if the measured level of LCN2 is below a control level or treating the patient with IL-22, a LCN2 inhibitor and/or an AHR agonist if the measured level of LCN2 is above the control level. The treatment can further comprise an antibiotic.

In certain embodiments, the sample is a stool sample. In other embodiments, the sample is a blood sample. In particular embodiments, step (a) is accomplished using a polymerase chain reaction (PCR) assay. In a more specific embodiment, the PCR assay uses a primer comprising SEQ ID NO:3 and/or SEQ ID NO:4. In alternative embodiments, step (a) is accomplished using an immunoassay.

In a further embodiments, the patient undergoes a fecal transplant if the measured level of LCN2 is above the control level. In yet another embodiment, the LCN2 inhibitor is a small molecule, an antibody or an inhibitory nucleic acid molecule. In more specific embodiments, the inhibitory nucleic acid molecule is a siRNA, shRNA, antisense RNA or a ribozyme.

In another aspect, the present invention provides a PCR assay for measuring LCN2.

In one embodiment, a method comprises performing a PCR assay to measure LCN2 in a sample obtained from a patient having SBS. In a specific embodiment, the PCR assay uses a primer comprising SEQ ID NO:3 and/or SEQ ID NO:4.

In a further aspect, the present invention provides compositions and methods for treating conditions associated with SBS including, but not limited to, intestinal dysbiosis. In a specific embodiment, a method for treating intestinal dysbiosis associated with SBS in a patient comprises the step of administering to the patient IL-22, a LCN2 inhibitor and/or an AHR agonist. In particular embodiments, the LCN2 inhibitor is a small molecule, an antibody or an inhibitory nucleic acid molecule. In more specific embodiments, the inhibitory nucleic acid molecule is a siRNA, shRNA, antisense RNA or a ribozyme. In more specific embodiments, the treatment further comprises an aryl hydrocarbon receptor (AHR) agonist.

In yet another aspect, the present invention also provides compositions and methods for assessing intestinal adaptation of a patient having SBS. In a specific embodiment, a method comprises the steps of (a) measuring LCN2 in a sample obtained from the patient; and (b) comparing the measured LCN2 of step (a) to a control level, wherein LCN2 expression below the control level correlates with intestinal adaptation, and wherein LCN2 expression above the control level does not correlate with intestinal adaptation. In certain embodiments, the patient whose LCN2 expression correlates with intestinal adaptation reduces or eliminates parenteral nutrition. In other words, if a level of LCN2 decreases, then a doctor could reduce the amount of parenteral nutrition because they would be confident that adaptation was progressing. On the other hand, should the LCN2 levels increase, then a decrease in adaptation would be the conclusion, and the provider would increase the parenteral nutrition. In other embodiments, the patient whose LCN2 expression does not correlate with intestinal adaptation undergoes one or more of IL-22 treatment, LCN2 inhibitor treatment, AHR agonist treatment, and a fecal transplant. In particular embodiments, the LCN2 inhibitor is a small molecule, an antibody or an inhibitory nucleic acid molecule. In more specific embodiments, the inhibitory nucleic acid molecule is a siRNA, shRNA, antisense RNA or a ribozyme. In particular embodiments, the sample is a blood, serum or stool sample.

### BRIEF DESCRIPTION OF THE FIGURES

FIG. 1A-1F. Mice undergoing 75% SBR showed significant intestinal adaptation.

FIG. 1A: Body weight changes in SHA (n=6) and 75% SBR (n=6) mice from post-operative day (POD) 1 to POD 7. FIG. 1B: Enterocyte proliferation were assessed by BrdU staining on POD 7. FIG. 1C: Intestinal permeability was evaluated by the fold change of FITC-Dextran in serum on POD 7. FIG. 1D: Representative H&E stained sections ofjejunum and ileum 7 days after sham or SBR surgery. Scale bar for jejunum is 1000 μm and ileum is 500 μm. Length of villi in jejunum (FIG. 1E) and ileum (FIG. 1F) were measured under light microscopy. At least 30 well-oriented intact villi were counted per mouse. Each dot represented a mouse. SHA, sham; SBR, small bowel resection. ***P<0.001; #P<0.05, ##P<0.01, ###P<0.001 vs. WT SHA respectively.

FIG. 2A-2I. Intestinal dysbiosis in WT mice after sham or 75% SBR. Intestinal microbiome was evaluated by 16S rRNA sequencing on post-operative 1 week and 3 weeks. Taxa richness and evenness were evaluated by (FIG. 2A) Chao 1 richness, (FIG. 2B) Faith' diversity, and (FIG. 2C) total OTUs. Relative abundance of (FIG. 2D) Proteobacteria (FIG. 2E) Bacteroidetes, (FIG. 2F) Firmicutes (FIG. 2G) Enterococcus, (FIG. 2H) Clostridium were assessed in sham and 75% SBR groups. Beta-diversity was shown in (FIG. 2I) PCoA plot. WT SHA post 1 week n=9, WT SBR post 1 week n=10, WT SHA post 3 weeks n=8, WT SBR post 3 weeks n=7. Each dot represented a mouse. SHA, sham; SBR, small bowel resection. *P<0.05, **P<0.01, ***P<0.001.

FIG. 3A-3G. LCN2 expression significantly increased in WT mice after 75% SBR on post-operative day 7. LCN2 expression in the serum (FIG. 3A) and feces (FIG. 3B) was measured by ELISA (n=6 per group). Transcription levels of the Lcn2 gene (FIG. 3C) in the intestine and liver were evaluated by quantitative q-PCR (n=5 per group). Representative LCN2 protein expression (FIG. 3D-3E) in the intestine and liver were measured with western blot analysis (n=4 per group). FIG. 3F: Representative LCN2-stained sections of jejunum after sham or SBR surgery (n=3 per group). FIG. 3G: Representative LCN2, MPO, and F4/80-co-stained sections of jejunum after SBR surgery (n=3). Each dot represented a mouse. SHA, sham; SBR, small bowel resection. **P<0.01, ***P<0.001; #P<0.05, ##P<0.01, ###P<0.001 vs. WT SHA respectively.

FIG. 4A-4H. Depletion of intestinal microbiota decreased intestinal adaptation in SBS. FIG. 4A: Survival rate was evaluated from POD 1 to POD 7, CTL SHA (n=6), CTL SBR (n=8), ABX-SHA (n=6), ABX-SBR (n=8). FIG. 4B: Body weight changes in CTL SHA (n=6), CTL SBR (n=7), ABX-SHA (n=6), ABX-SBR (n=3) from POD 1 to POD 7. FIG. 4C: Length of villi in jejunum were measured under light microscopy. At least 30 well-oriented intact villi were counted per mouse. FIG. 4D: Fecal bacterial load was evaluated by q-PCR with universal 16S primers. LCN2 expression in jejunum (FIGS. 4E & 4G) and liver (FIGS. 4F & 4H) following antibiotic treatment were measured by western blot analysis. Each dot represented a mouse. SHA, sham; SBR, small bowel resection; ABX, antibiotics. *P<0.05, **P<0.01, ***P<0.001; #P<0.05, ##P<0.01.

FIGS. 5A-5J. LCN2^{-/-} mice had less intestinal inflammation and greater functional adaptation following 75% SBR than WT mice. FIG. 5A: Body weight changes in WT and LCN2^{-/-} mice after sham operation and 75% SBR from POD 1 to POD 7 (n=10 per group). FIG. 5B: Intestinal permeability was evaluated by the fold-change of FITC-Dextran in serum on POD 7 (n=10 per group). FIG. 5C: Sucrase-isomaltase were quantified by q-PCR (n=5 per group). FIG. 5D: Representative H&E stained sections of jejunum and ileum 7 days after sham or SBR surgery. Scale bar for jejunum is 1000 μm and ileum is 500 μm. Length of villi in jejunum (FIG. 5E) and ileum (FIG. 5F) were measured under light microscopy (n=10 per group). At least 30 well-oriented intact villi were counted per mouse. Transcription levels of (FIG. 5G) Tgr5, (FIG. 5H) Tnf-α, and (FIG. 5I) IL-6 genes in small intestine were evaluated by quantitative q-PCR (n=6 per group). A systemic feature of inflammation, (FIG. 5J) IL-6 in serum was measured by ELISA (n=10 per group). Each dot represented a mouse. SHA, sham; SBR, small bowel resection. **P<0.01, ***P<0.001; #P<0.05, ##P<0.01, ###P<0.001 vs. WT SBR respectively.

FIG. 6A-6I. Intestinal microbiome changes in WT and LCN2^{-/-} mice following sham or 75% SBR. Intestinal microbiome was evaluated by 16S rRNA sequencing on POD 7. Taxa richness and evenness were evaluated by (FIG. 6A) Chao 1 richness, (FIG. 6B) Faith' diversity, and (FIG. 6C) total OTUs. FIG. 6D: Beta-diversity was shown in PCoA plot (each dot represented a mouse). KEGG pathways at level 3 for gut microbiome of primary (FIG. 6E) and secondary (FIG. 6F) bile acid biosynthesis. Relative abundance of (FIG. 6G) Proteobacteria (FIG. 6H) Bacteroidetes, and (FIG. 6I) Firmicutes. WT SHA n=8, WT SBR n=8, LCN2^{-/-} SHA n=8, LCN2^{-/-} SBR n=9. SHA, sham; SBR, small bowel resection. *P<0.05, **P<0.01, ***P<0.001.

FIG. 7A-7E. Intestinal adaptation in germ-free mice after fecal microbiota transplantation. The 4% fecal slurry were created from cecal contents of conventional WT SBR and LCN2^{-/-} SBR mice respectively and were gavage fed (100 μL/mouse) to germ-free mice. FIG. 7A: Body weight changes in germ-free mice transplanted with fecal contents from WT SBR or LCN2^{-/-} SBR mice on post-transplant day 7. FIG. 7B: Intestinal permeability was evaluated by the fold change of FITC-Dextran in serum on post-transplant day 7. FIG. 7C: Representative H&E stained sections of jejunum and ileum 7 days after fecal microbiota transplantation. Scale bar for jejunum and ileum is 500 μm. Length of villi in jejunum (FIG. 7D) and ileum (FIG. 7E) were measured under light microscopy. At least 30 well-oriented intact villi were counted per germ-free mouse. GWR (n=6), germ-free mice received fecal matters from WT SBR donor; GLR (n=6), germ-free mice received fecal matters from LCN2^{-/-} SBR donor. Each dot represented a mouse *P<0.05, **P<0.01, ***P<0.001.

FIG. 8A-8E. IL-22^{-/-} mice had worse intestinal adaptation with less weight gain and shorter jejunal villi following 75% SBR than WT mice. FIG. 8A: Body weight changes in WT and IL-22^{-/-} mice after sham operation and 75% SBR from POD 1 to POD 7 (n=3 per group). FIG. 8B: Intestinal permeability was evaluated by the fold-change of FITC-Dextran in serum on POD 7 (n=3 per group). FIG. 8C: Representative H&E stained sections of jejunum and ileum 7 days after sham or SBR surgery. Scale bar for jejunum is 1000 μm and ileum is 500 μm. Length of villi in jejunum (FIG. 8D) and ileum (FIG. 8E) were measured under light microscopy (n=3 per group). At least 30 well-oriented intact villi were counted per mouse. Each dot represented a mouse. *P<0.05, **P<0.01, ***P<0.001; #P<0.05, ##P<0.01.

FIG. 9A-9H. LCN2 reduced intestinal adaptation by inhibiting IL-22 expression following 75% SBR on post-operative day 7. FIG. 9A; Transcriptional level of IL-22 gene in intestine was evaluated by quantitative q-PCR (n=6 per group). FIG. 9B: IL-22 protein expression in serum was measured by ELISA (n=7 per group). FIG. 9C: CD4+IL-22+LPLs were quantified with flow cytometry analysis (n=5 per group). FIG. 9D: Representative IL-22 protein expression in supernatant from Th22 cells in vitro with and without LCN2 stimulation as measured by ELISA (n=3 per group/each experiment, experiment was repeated three times). (FIG. 9E-9H) Representative flow cytometry dot plots of lamina propria lymphocyte (LPL) population from small intestinal tissue. Single cell suspensions prepared from lamina propria were stained with CD4 and IL-22 antibodies. Gate 1 identified lymphocytes based on FSC-A/SSC-A properties. Numbers within the quadrants represented the percentage of CD4⁺ IL-22⁻, CD4⁺ IL-22⁺, CD4⁻ IL-22⁻, CD4⁻ IL-22⁺ cells within the lymphocytes gate. Data are representative of analyses of 5 mice per group. *P<0.05, **P<0.01, ***P<0.001.

FIG. 10A-10J. IL-22 promoted intestinal adaptation and counteracted dysbiosis following 75% SBR. FIG. 10A: Body weight changes in WT mice treated with either PBS (n=3) or recombinant mouse IL-22 (rmIL-22) (n=4) after 75% SBR from POD 1 to POD 7. Length of villi in jejunum (FIG. 10B) and ileum (FIG. 10C) was measured under light microscopy. At least 30 well-oriented intact villi were counted per mouse. Transcriptional levels of IL-22 mediated anti-bacterial peptide (FIG. 10D) Reg3b and (FIG. 10E) Reg3g in jejunum, and (FIG. 10F) Reg3b and (FIG. 10G) Reg3g in colon, (FIG. 10J) Tnf-α in jejunum were measured by quantitative q-PCR. Relative abundance of (FIG. 10H) Proteobacteria, (FIG. 10I) Bacteroidetes, Firmicutes were evaluated by quantitative q-PCR. Each dot represented a mouse. *P<0.05, **P<0.01, ***P<0.001; #P<0.05.

FIG. 11A-11F. Lipocalin 2 as a Marker of Inflammation and Decreased Adaptation in Short Bowel Syndrome. Transcriptional levels of (FIG. 11A) Tnf-α, (FIG. 11B) Mki67, (FIG. 11C) Puma, (FIG. 11D) sucrase-isomaltase, and (FIG. 11E) Muc2 in small intestine from wild-type and LCN2^{-/-} mice were evaluated by q-PCR on post-operative day 21. (FIG. 11F) Human LCN2 in feces levels were quantified by ELISA. *p<0.05, **p<0.01, ***p<0.001.

FIG. 12. Graphical schematic showing normal intestinal milieu and short bowel syndrome.

FIG. 13. I3C supplementation, an AHR agonist, increases weight gain in mice following SBR. Note the flat weight gain between postoperative days 3-7 in control DMSO-treated WT SBR mice (shown in black) vs. the significant increase in weight gain in I3C-treated WT SBR mice (shown in blue) *p=0.04 at day 7.

FIG. 14. I3C supplementation, an AHR agonist, increases weight gain in mice following SBR.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

In Short Bowel Syndrome (SBS), inadequate intestinal adaptation is responsible for the majority of complications, including sepsis, liver failure and death. The present inventors sought to further delineate the adaptive response to identify potential therapeutic targets. The present inventors performed a 75% small bowel resection (SBR) or sham operation on C57Bl/6J wild-type, lipocalin-2 (LCN2)^{-/-} and Interleukin-22 (IL-22)^{-/-} mice. Exogenous IL-22 was administered to SBR WT mice. Cecal fecal matter from SBR WT and SBR LCN2^{-/-} mice were transplanted into germ-free mice. Intestinal permeability, inflammation, proliferation and microbiome were evaluated one week following surgery. CD4⁺IL-22⁺ laminal propria lymphocytes were sorted by flow cytometry. Naïve T cells were polarized to Th22 cells with or without LCN2.

Seventy-five percent SBR in a mouse recreates the increased intestinal permeability, enterocyte proliferation and intestinal dysbiosis seen in SBS. LCN2 expression increases following 75% SBR, and this increase can be abrogated with broad-spectrum antibiotic treatment. LCN2^{-/-} mice have less intestinal inflammation, increased IL-22 expression, less intestinal permeability, increased carbohydrate enzyme expression, less weight loss and less dysbiosis following 75% SBR than WT mice. The pro-inflammatory and anti-adaptive effects of LCN2 can be transferred to germ-free mice via a fecal transplant. Administration of exogenous IL-22 improves adaptation and restores the normal microbiome following 75% SBR in WT mice.

Accordingly, LCN2 promotes inflammation and retards intestinal adaptation through changes in the microbiome and IL-22 inhibition in a mouse model of SBS. Strategies to reduce LCN2 may offer novel therapeutic approaches to enhance adaptation in SBS.

### I. Definitions

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%. An alteration may be, for example, a change in expression level or activity.

By “control” is meant a standard or reference condition. For example, LCN2 expression in a sample from a patient having SBS may be compared to the level of LCN2 expression from the same patient at an earlier time, from a patient showing intestinal adaptation, from a patient not having SBS, and the like.

“Detect” refers to identifying the presence, absence or amount of the analyte to be detected. In certain embodiments, “detect” is used interchangeably with “measure.”

The term “nucleotide sequence” refers to a polymer of DNA or RNA which can be single-stranded or double-stranded, optionally containing synthetic, non-natural or altered nucleotide bases capable of incorporation into DNA or RNA polymers. A DNA molecule or polynucleotide is a polymer of deoxyribonucleotides (A, G, C, and T), and an RNA molecule or polynucleotide is a polymer of ribonucleotides (A, G, C and U).

The terms “complementary” and “complementarity” refer to polynucleotides (i.e., a sequence of nucleotides) related by the base-pairing rules. For example, the sequence “A-G-T”, is complementary to the sequence “T-C-A.” Complementarity may be “partial”, in which only some of the nucleic acids' bases are matched according to the base pairing rules. Alternatively, there may be “complete” or “total” complementarity between the nucleic acids. The degree of complementarity between nucleic acid strands has significant effects on the efficiency and strength of hybridization between nucleic acid strands.

A “gene,” for the purposes of the present invention, includes a DNA region encoding a gene product, as well as all DNA regions which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses mRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

“Gene expression” refers to the conversion of the information contained in a gene into a gene product. It refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein. The term “altered level of expression” refers to the level of expression in cells or organisms that differs from that of normal cells or organisms.

A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase catalyzed transcription of a DNA sequence. When the RNA transcript is a perfect, complementary copy of the DNA sequence, it is referred to as the primary transcript or it may be a RNA sequence derived from post-transcriptional processing of the primary transcript and is referred to as the mature RNA “Messenger RNA” (mRNA) refers to the RNA that is without intrans and that can be translated into protein by the cell. “cDNA” refers to a single- or a double-stranded DNA that is complementary to and derived from mRNA. “Functional RNA” refers to sense RNA, antisense RNA, ribozyme RNA, siRNA, or other RNA that may not be translated but yet has an effect on at least one cellular process.

A “coding sequence,” or a sequence that “encodes” a selected polypeptide, is a nucleic acid molecule that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and especially synthetic DNA sequences. A transcription termination sequence may be located 3′ to the coding sequence.

Certain embodiments of the disclosure encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The terms “LCN2 expression” refers to the transcription and/or translation and/or activity of LCN2. Several methods can be utilized to determine the level of expression, as described in detail below. Expression from the human LCN2 gene results in the transcript set forth in SEQ ID NO: 33 (represented as coding sequence/cDNA) and the LCN2 protein set forth in SEQ ID NO: 34. The human LCN2 gene sequence is set forth in SEQ ID NO: 35.

The term “sample” as used herein refers to any biological specimen that may be extracted, untreated, treated, diluted or concentrated from a subject. The sample can be a biological sample. The biological samples are generally derived from a patient, including a cell sample or bodily fluid (such as tumor tissue, lymph node, sputum, blood, bone marrow, cerebrospinal fluid, phlegm, saliva, or urine) or cell lysate. The cell lysate can be prepared from a tissue sample (e.g., a tissue sample obtained by biopsy), for example, a tissue sample (e.g., a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In specific embodiments, the sample is a stool sample. In other embodiments, the sample is blood.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

“Protein,” “polypeptide” and “peptide” are used interchangeably herein to refer to a polymer of amino acid residues and to variants and synthetic analogues of the same.

The term “antibody” and its grammatical equivalents refer to a protein which is capable of specifically binding to a target antigen and includes any substance, or group of substances, which has a specific binding affinity for an antigen, suitably to the exclusion of other substances. This term encompasses an immunoglobulin molecule capable of specifically binding to a target antigen by virtue of an antigen binding site contained within at least one variable region. This term includes four chain antibodies (e.g., two light chains and two heavy chains), recombinant or modified antibodies (e.g., chimeric antibodies, humanized antibodies, primatized antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)).

In one example, the antibody is a murine (mouse or rat) antibody or a primate (suitably human) antibody. The term “antibody” encompasses not only intact polyclonal or monoclonal antibodies, but also variants, fusion proteins comprising an antibody portion with an antigen binding site, humanized antibodies, human antibodies, chimeric antibodies, primatized antibodies, de-immunized antibodies or veneered antibodies. Also within the scope of the term “antibody” are antigen-binding fragments that retain specific binding affinity for an antigen, suitably to the exclusion of other substances. This term includes a Fab fragment, a Fab′ fragment, a F(ab′) fragment, a single chain antibody, and the like.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

The terms “marker” or “biomarker” are used interchangeably, broadly refer to any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular antibody can be indicative of the presence of a particular antigen (e.g., protein) in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional.

The “level”, “abundance” or “amount” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment. In specific embodiments in which the level of a biomarker (e.g., LCN2) is “reduced” relative to a reference or control, the reduced level may refer to an overall reduction of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the decrease is at least about any of at least 0.9×, 0.8×, 0.7×, 0.6×, 0.5×, 0.4×, 0.3×, 0.2×, 0.1, 0.05×, 0.01×, and the like, the level/amount of the respective biomarker in a reference sample, reference cell, reference tissue, control sample, control cell, or control tissue. In specific embodiments in which the level of a biomarker (e.g., LCN2) is “increased” relative to a reference or control, the reduced level may refer to an overall increase of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the increase is at least about any of at least about 1.1×, 1.2×, 1.3×, 1.4×, 1.5×, 1.6×, 1.7×, 1.8×, 1.9×, 2×, 3×, 4×, 5×, 10×, 20×, 30×, 50×, 80×, 100×, and the like, the level/amount of the respective biomarker in a reference sample, reference cell, reference tissue, control sample, control cell, or control tissue. In certain embodiments in which the level of a biomarker is “about the same” a reference or control, the level of biomarker varies by less than about 10%, 9%, 8%, 7%, 6%, 5%, 4%, 3%, 2%, 1%, 0.5%, 0.1%, or even less, as compared to the level of biomarker (e.g., protein or nucleic acid (e.g., mRNA or cDNA)), detected by standard art known methods such as those described herein, in a reference sample, reference cell, reference tissue, control sample, control cell, or control tissue.

The term “indicator” as used herein refers to a result or representation of a result, including any information, number, ratio, signal, sign, mark, or note by which a skilled artisan can estimate and/or determine a likelihood of whether or not a subject has a condition or a subject suffering from the condition will respond to a relevant therapy. An “indicator” may optionally be used together with other clinical characteristics to arrive at a determination that the subject has a condition or is or is not likely to respond to a therapy. That such an indicator is “determined” is not meant to imply that the indicator is 100% accurate. The skilled clinician may use the indicator together with other clinical indicia to arrive at a conclusion.

As used herein, the term “label” and grammatical equivalents thereof, refer to any atom or molecule that can be used to provide a detectable and/or quantifiable signal. In particular, the label can be attached, directly or indirectly, to a nucleic acid or protein. Suitable labels that can be attached include, but are not limited to, radioisotopes, fluorophores, quenchers, chromophores, mass labels, electron dense particles, magnetic particles, spin labels, molecules that emit chemiluminescence, electrochemically active molecules, enzymes, cofactors, and enzyme substrates. A label can include an atom or molecule capable of producing a visually detectable signal when reacted with an enzyme. In some embodiments, the label is a “direct” label which is capable of spontaneously producing a detectible signal without the addition of ancillary reagents and is detected by visual means without the aid of instruments. For example, colloidal gold particles can be used as the label. Many labels are well known to those skilled in the art. In specific embodiments, the label is other than a naturally-occurring nucleoside. The term “label” also refers to an agent that has been artificially added, linked or attached via chemical manipulation to a molecule.

As used herein, “primer,” “probe,” and “oligonucleotide” are used interchangeably.

The term “nucleic acid probe” or a “probe specific for” a nucleic acid refers to a nucleic acid sequence that has at least about 80%, e.g., at least about 90%, e.g., at least about 95%, 96%, 97%, 98%, 99% contiguous sequence identity or homology to the nucleic acid sequence encoding the targeted sequence of interest. A probe (or oligonucleotide or primer) of the disclosure is at least about 8 nucleotides in length (e.g., at least about 8-50 nucleotides in length, e.g., at least about 10⁻⁴⁰, e.g., at least about 15-35 nucleotides in length). The oligonucleotide probes or primers of the disclosure may comprise at least about eight nucleotides at the 3′ of the oligonucleotide that have at least about 80%, e.g., at least about 85%, e.g., at least about 90% contiguous identity to the targeted sequence of interest.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded; if double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including application, temperature to be employed, template reaction conditions, other reagents, and source of primers. The oligonucleotide primer typically contains 12-20 or more nucleotides, although it may contain fewer nucleotides. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide.

In certain embodiments, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physicochemical interactions typically occur in interactions between molecules. In particular embodiments, all that is required is that the molecules (e.g., nucleic acids or polypeptides) remain immobilized or attached to a support under conditions in which it is intended to use the support, for example in applications requiring nucleic acid amplification and/or sequencing or in in antibody-binding assays. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in a subject's feces. A non-selective fecal microbiota refers to a community or mixture of fecal microbes derived from a donor's fecal sample without selection and substantially resembling microbial constituents and population structure found in such fecal sample.

As used herein, the term “fecal microbes” refers to microorganisms that are present in the gut, intestine, or colon, preferably colon, or feces of a donor such as a normal healthy adult human.

As used herein, the term “fecal material” refers to human stool. Unprocessed fecal material contains nonliving material and biological material.

The term “non-living material” refers to the non-living material in fecal material, and may include, but is not limited to, dead bacteria, shed host cells, proteins, carbohydrates, fats, minerals, mucus, bile, undigested fiber and other foods, and other compounds resulting from food and metabolic ingestion and waste products and partial or complete digestion of food materials.

“Biological material” refers to the living material in fecal material, and includes microbes including prokaryotic cells, such as bacteria and archea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human.

As used herein, “antibiotic” refers to a substance that is used to treat and/or prevent bacterial infection by killing bacteria, inhibiting the growth of bacteria, or reducing the viability of bacteria.

As used herein, the terms “diagnosis,” “diagnosing” and the like are used interchangeably herein to encompass determining the likelihood that a subject will develop or has a condition or clinical state. These terms also encompass, for example, determining the level of clinical state (e.g., the level of responsiveness to a therapy), as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or derived biomarker values actually has a condition or clinical state (or not) based on a given mathematical model. An increased likelihood for example may be relative or absolute and may be expressed qualitatively or quantitatively. For instance, an increased likelihood may be determined simply by determining the subject's measured LCN2 levels and placing the subject in an “increased likelihood” category, based upon previous population studies. The term “likelihood” is also used interchangeably herein with the term “probability”.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, the term “positive response” means that the result of a treatment regimen includes some clinically significant benefit, such as the prevention, or reduction of severity, of symptoms, or a slowing of the progression of the condition. By contrast, the term “negative response” or “non-response” means that a treatment regimen provides no or minimal clinically significant benefit, such as the prevention, or reduction of severity, of symptoms, or increases the rate of progression of the condition.

The term “prognosis” as used herein refers to a prediction of the probable course and outcome of a clinical condition or disease. A prognosis is usually made by evaluating factors or symptoms of a disease or condition that are indicative of a favorable or unfavorable course or outcome of the disease or condition (e.g., response to therapy). The skilled artisan will understand that the term “prognosis” refers to an increased probability that a certain course or outcome will occur; that is, that a course or outcome is more likely to occur in a subject exhibiting a given condition, when compared to those individuals not exhibiting the condition.

As used herein, the term “intestinal adaptation” should be understood to encompass any physiological change by which the nutrient and fluid absorptive capacity of the small intestine is enhanced, increased, grown, supported or advanced including, but not limited to, enlargement or lengthening of the villi found in the lining, increase in the diameter of the small intestine and reduction in peristalsis or movement of food through the small intestine.

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that down-regulates (e.g., suppresses or inhibits) at least one bioactivity of a protein such as LCN2. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. The term“inhibitor” is synonymous with the term antagonist. The term also includes agents that have activity in addition to LCN2 inhibitory activity.

An “agonist” is a type of modulator and refers to an agent that can activate one or more functions of a target. For example, an agonist of a protein can activate the protein in the absence of its natural or cognate ligand.

A“small molecule” refers to a composition that has a molecular weight of less than 3 about kilodaltons (kDa), less than about 1.5 kilodaltons, or less than about 1 kilodalton. In particular embodiments, small molecules may be nucleic acids, peptides, polypeptides, peptidomimetics, carbohydrates, lipids or other organic (carbon-containing) or inorganic molecules. In some embodiments, a “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “LCN2 modulator” refers to an agent that modulates the expression and/or activity of LCN2. Inhibitors may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is preferably useful in the treatment of a disease disclosed herein.

The use of the term “in combination” does not restrict the order in which the therapies (e.g., LCN2 inhibitor, IL-22 and/or AHR agonist) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a patient who has SBS. The therapies are administered to a patient in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The terms “synergy,” “synergistic,” or “synergistic effect” as used herein describe an effect that has a magnitude that is greater than the sum if the individual effects. In some embodiments of the present invention, the use of an LCN2 inhibitor, IL-22 and/or an AHR agonist in concert provides a synergistic therapeutic effect on a neoplastic condition in a patient and/or on the growth of a cell.

Optional” or “optionally” means that the subsequently described event or circumstance can or cannot occur, and that the description includes instances where the event or circumstance occurs and instances where it does not.

It will be appreciated that the terms used herein and associated definitions are used for the purpose of explanation only and are not intended to be limiting.

### II. Sample Preparation for Detection of LCN2 Expression

In certain embodiments, a sample from a subject with SBS is processed prior to LCN2 detection or quantification. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide detection (e.g., quantification of RNA or protein biomarkers) from raw sample (e.g., biological fluid such as blood, serum, etc.) without or with limited processing. In some examples, whole cells or tissue sections are isolated and analyzed for LCN2 expression such as using immunohistochemistry (IHC) or flow cytometry.

Methods may comprise steps of homogenizing a sample in a suitable buffer, removal of contaminants and/or assay inhibitors, adding a LCN2 capture reagent (e.g., a magnetic bead to which is linked an oligonucleotide complementary to a LCN2 biomarker), incubated under conditions that promote the association (e.g., by hybridization) of the target biomarker with the capture reagent to produce a target biomarker:capture reagent complex, incubating the target biomarker:capture complex under target biomarker-release conditions. In some embodiments, multiple biomarkers are isolated in each round of isolation by adding multiple biomarkers capture reagents (e.g., specific to the desired biomarkers) to the solution. For example, multiple biomarker capture reagents, each comprising an oligonucleotide specific for a different biomarker can be added to the sample for isolation/detection/measurement of multiple biomarkers. It is contemplated that the methods encompass multiple experimental designs that vary both in the number of capture steps and in the number of target biomarkers captured in each capture step.

In some embodiments, capture reagents are molecules, moieties, substances, or compositions that preferentially (e.g., specifically and selectively) interact with a particular biomarker sought to be isolated, purified, detected, and/or quantified. Any capture reagent having desired binding affinity and/or specificity to the particular biomarker can be used in the present technology.

For example, the LCN2 capture reagent can be a macromolecule such as a peptide, a protein (e.g., an antibody or other ligand that specifically binds to LCN2), an oligonucleotide, a nucleic acid (e.g., nucleic acids capable of hybridizing with LCN2), oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary polynucleotide.

Any nucleic acids, including single-stranded and double-stranded nucleic acids, that are capable of binding, or specifically binding, to a target LCN2 biomarker can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, LCN2 biomarker capture reagents may comprise a functionality to localize, concentrate, aggregate, etc., the capture reagent and thus provide a way to isolate and purify the target biomarker when captured (e.g., bound, hybridized, etc.) to the capture reagent (e.g., when a target:capture reagent complex is formed). For example, in some embodiments the portion of the capture reagent that interacts with the biomarker (e.g., an oligonucleotide) is linked to a solid support (e.g., a bead, surface, resin, column, and the like) that allows manipulation by the user on a macroscopic scale. Often, the solid support allows the use of a mechanical means to isolate and purify the target:capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target biomarker) from the heterogeneous solution.

The LCN2 biomarker may be quantified or detected using any suitable technique. In specific embodiments, the LCN2 biomarker is quantified using reagents that determine the level, abundance or amount of the individual biomarker, either as isolated biomarker or as expressed in or on a cell. Non-limiting reagents of this type include reagents for use in nucleic acid- and protein-based assays, as described below.

### III. Detection of LCN2 Nucleic Acid

Many methods of measuring the levels or amounts of biomarker nucleic acid expression, including LCN2, are contemplated. Any reliable, sensitive, and specific method can be used. In particular embodiments, biomarker nucleic acid is amplified prior to measurement. In other embodiments, the level of biomarker nucleic acid is measured during the amplification process. In still other methods, the target nucleic acid is not amplified prior to measurement.

A. Amplification Reactions

Many methods exist for amplifying nucleic acid sequences. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR).

A typical PCR reaction comprises multiple amplification steps or cycles that selectively amplify target nucleic acid species including a denaturing step in which a target nucleic acid is denatured; an annealing step in which a set of PCR primers (forward and reverse primers) anneal to complementary DNA strands; and an extension step in which a thermostable DNA polymerase extends the primers. By repeating these steps multiple times, a DNA fragment is amplified to produce an amplicon, corresponding to the target DNA sequence. Typical PCR reactions include about 20 or more cycles of denaturation, annealing, and extension. In many cases, the annealing and extension steps can be performed concurrently, in which case the cycle contains only two steps. Because mature mRNA are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than about 15, fewer than about 20, fewer than about 25, fewer than about 30, or fewer than about 35 nucleotides in length. In additional embodiments, a primer is at least about 35 nucleotides in length.

In a further embodiment, a forward primer can comprise at least one sequence that anneals to biomarker nucleic acid sequence and alternatively can comprise an additional 5′ non-complementary region. In another embodiment, a reverse primer can be designed to anneal to the complement of a reverse transcribed mRNA. The reverse primer may be independent of the biomarker nucleic acid sequence, and multiple biomarker nucleic acid sequences may be amplified using the same reverse primer. Alternatively, a reverse primer may be specific for a biomarker nucleic acid.

In some embodiments, two or more biomarker nucleic acid sequences (e.g., LCN2 and another marker) are amplified in a single reaction volume. One aspect includes multiplex q-PCR, such as qRT-PCR, which enables simultaneous amplification and quantification of at least two biomarker nucleic acid sequences of interest in one reaction volume by using more than one pair of primers and/or more than one probe. The primer pairs comprise at least one amplification primer that uniquely binds each mRNA, and the probes are labeled such that they are distinguishable from one another, thus allowing simultaneous quantification of multiple biomarker nucleic acid sequences. Multiplex qRT-PCR has research and diagnostic uses including, but not limited, to detection of biomarker nucleic acid sequences for diagnostic, prognostic, and therapeutic applications.

The qRT-PCR reaction may further be combined with the reverse transcription reaction by including both a reverse transcriptase and a DNA-based thermostable DNA polymerase. When two polymerases are used, a “hot start” approach may be used to maximize assay performance. See U.S. Pat. Nos. 5,985,619 and 5,411,876. For example, the components for a reverse transcriptase reaction and a PCR reaction may be sequestered using one or more thermoactivation methods or chemical alteration to improve polymerization efficiency. See U.S. Pat. Nos. 6,403,341; 5,550,044; and 5,413,924.

B. Detection Reactions

In certain embodiments, labels, dyes, or labeled probes and/or primers are used to detect amplified or unamplified LCN2 biomarker nucleic acid sequence (mRNA/cDNA). One of ordinary skill in the art will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where biomarker nucleic acid sequence amplification is preferred.

A probe or primer may include Watson-Crick bases or modified bases. Modified bases include, but are not limited to, the AEGIS bases (from EraGen Biosciences, Inc. (Madison, Wis.)), which have been described, e.g., in U.S. Pat. Nos. 6,001,983; 5,965,364; and 5,432,272. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan®) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,649,349 and 6,485,901), peptide nucleic acid (PNA) Molecular Beacons (see, e.g., U.S. Pat. Nos. 6,593,091 and 6,355,421), linear PNA beacons (see, e.g., U.S. Pat. No. 6,329,144), non-FRET probes (see, e.g., U.S. Pat. No. 6,150,097), Sunrise®/Amplifluor® probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse® probe (Sigma-Aldrich Corp. (St. Louis, Mo.)), hairpin probes (see, e.g., U.S. Pat. No. 6,596,490), PNA light-up probes, antiprimer quench probes (Li et al., 53 CLIN. CHEM. 624-33 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Biomarker nucleic acid sequences can be detected by direct or indirect methods. In a direct detection method, one or more biomarker nucleic acid sequences are detected by a detectable label that is linked to a nucleic acid molecule. In such methods, the biomarker nucleic acid sequences may be labeled prior to binding to the probe. Therefore, binding is detected by screening for the labeled biomarker nucleic acid sequence that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified mRNA/cDNA, are detected using xMAP Microspheres (Luminex Corp. (Austin, Tex.)) conjugated with probes to capture the desired nucleic acids. Some methods may involve detection with polynucleotide probes modified, for example, with fluorescent labels or branched DNA (bDNA) detection.

In other embodiments, nucleic acids are detected by indirect detection methods. For example, a biotinylated probe may be combined with a stretavidin-conjugated dye to detect the bound nucleic acid. The streptavidin molecule binds a biotin label on amplified nucleic acid, and the bound nucleic acid is detected by detecting the dye molecule attached to the streptavidin molecule. In one embodiment, the streptavidin-conjugated dye molecule comprises Phycolink® Streptavidin R-Phycoerythrin (ProZyme, Inc. (Heward, Calif.)). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal. See, e.g., Garman A., Non-Radioactive Labeling, Academic Press (1997) and Kricka, L., Nonisotopic DNA Probe Techniques, Academic Press, San Diego (1992). Fluorescent reporter dyes useful as labels include, but are not limited to, fluoresceins (see, e.g., U.S. Pat. Nos. 6,020,481; 6,008,379; and 5,188,934), rhodamines (see, e.g., U.S. Pat. Nos. 6,191,278; 6,051,719; 5,936,087; 5,847,162; and 5,366,860), benzophenoxazines (see, e.g., U.S. Pat. No. 6,140,500), energy-transfer fluorescent dyes, comprising pairs of donors and acceptors (see, e.g., U.S. Pat. Nos. 5,945,526; 5,863,727; and 5,800,996; and), and cyanines (see, e.g., WO 9745539), lissamine, phycoerythrin, Cy2, Cy3, Cy3.5, Cy5, Cy5.5, Cy7, FluorX (Amersham Biosciences, Inc. (Piscataway, N.J.)), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4-tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VIC™, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In further embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify biomarker nucleic acid may be used including, but not limited to, oligonucleotide ligation (OLA) methods and methods that allow a distinguishable probe that hybridizes to the target nucleic acid sequence to be separated from an unbound probe. For example, HARP-like probes, as disclosed in U.S. Patent Application Publication No. 2006/0078894 may be used to measure the quantity of target nucleic acid. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify target biomarker nucleic acid. In a Multiplex Ligation-dependent Probe Amplification (MLPA) technique, pairs of probes which hybridize immediately adjacent to each other on the target nucleic acid are ligated to each other only in the presence of the target nucleic acid. See Schouten et al., 30 NUCL. ACIDS RES. e57 (2002). In some aspects, MLPA probes have flanking PCR primer binding sites. MLPA probes can only be amplified if they have been ligated, thus allowing for detection and quantification of biomarkers.

Furthermore, a sample may also be analyzed by means of a microarray. Microarrays generally comprise solid substrates and have a generally planar surface, to which a capture reagent (also called an adsorbent or affinity reagent) is attached. Frequently, the surface of a microarray comprises a plurality of addressable locations, each of which has the capture reagent (e.g., miRNA probes specific for particular biomarkers) bound there. Many microarrays are described in the art. These include, for example, biochips produced by Asuragen, Inc. (Austin, Tex.); Affymetrix, Inc. (Santa Clara, Calif.); GenoSensor Corp. (Tempe, Ariz.); Invitrogen, Corp. (Carlsbad, Calif.); and Illumina, Inc. (San Diego, Calif.). In certain embodiments, a target biomarker can be measured using a microfluidic card (e.g., TaqMan® Array Microfluidic Card (Applied Biosystems).

### IV. Detection of LCN2 Protein

A. Detection by Immunoassay

In specific embodiments, the target LCN2 biomarker of the present invention can be detected/measured by immunoassay. Immunoassay requires biospecific capture reagents/binding agent, such as antibodies, to capture the biomarker. Many antibodies are available commercially. Antibodies also can be produced by methods well known in the art, e.g., by immunizing animals with the biomarkers. Target biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the biomarkers employed herein are quantified by immunoassay, such as enzyme-linked immunoassay (ELISA) technology. In specific embodiments, the levels of expression of the biomarkers are determined by contacting the biological sample with antibodies, or antigen-binding fragments thereof, that selectively bind to the biomarker; and detecting binding of the antibodies, or antigen-binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety.

For example, the level of a LCN2 biomarker in a sample can be assayed by contacting the biological sample with an antibody, or antigen-binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. A target biomarker can be an entire protein, or a variant or modified form thereof. Kits for the detection of biomarkers as described herein can include pre-coated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, peptides, aptamer, etc., combinations thereof) to form a biomarker:capture agent complex. The complexes can then be detected and/or quantified.

In one method, a first capture or binding agent such as an antibody that specifically binds the biomarker of interest is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different polypeptide biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

B. Detection by Electrochemiluminescent Assay

In several embodiments, the LCN2 biomarker, and optionally other biomarkers, can be detected by means of an electrochemiluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

C. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the biomarkers comprising at least LCN2 described herein. In particular embodiments, a few micro-liters of blood serum or plasma are dropped on the chip array. Biomarker proteins present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### V. Kits for Detection of LCN2

All the essential reagents required for detecting and quantifying the LCN2 biomarker of the invention may be assembled together in a kit. In some embodiments, the kit comprises a reagent that permits quantification of LCN2. In some embodiments, the kit comprises: (i) at least one reagent that allows quantification (e.g., determining the abundance, concentration or level) of an expression product of LCN2 in a biological sample; and optionally (ii) instructions for using the at least one reagent. The kit can further comprise reagents for detection/measurement of other biomarkers including, but not limited to, AHR.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media, optical media, and the like. Alternatively, or in addition, the media can contain internet addresses that provide the instructions.

The kits may also optionally include appropriate reagents for detection of labels, positive and negative controls, washing solutions, blotting membranes, microtiter plates, dilution buffers and the like. For example, a protein-based detection kit may include an antibody that binds specifically to the LCN2 polypeptide. The kit may also include a LCN2 biomarker polypeptide to be used as positive control.

In particular embodiments, the kit is an immunoassay or ELISA kit. The ELISA kit may comprise a solid support, such as a chip, microtiter plate (e.g., a 96-well plate), bead, or resin having biomarker capture reagents attached thereon. The kit may further comprise a means for detecting the biomarker, such as antibodies, and a secondary antibody-signal complex such as horseradish peroxidase (HRP)-conjugated goat anti-rabbit IgG antibody and tetramethyl benzidine (TMB) as a substrate for HRP.

Alternatively, the kit may be provided as an immuno-chromatography strip comprising a membrane on which the antibodies are immobilized, and a detection agent, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be tested by adding a sample such as blood from the patient to the kit and detecting the relevant biomarker(s) conjugated with antibodies, specifically, by a method which comprises the steps of. (i) collecting a biological sample (e.g., blood) from the patient; (ii) adding the blood from the patient to a diagnostic kit; and, (iii) detecting the biomarker(s) conjugated with antibodies. In this method, the antibodies are brought into contact with the patient's blood. If the biomarker(s) are present in the sample, the antibodies will bind to the sample, or a portion thereof. In other kit and diagnostic embodiments, blood or blood serum need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

Alternatively, a nucleic acid-based detection kit may include a primer or probe that specifically hybridizes to a LCN2 polynucleotide (e.g., a cDNA of a LCN2 or LCN2 transcript or the transcript itself). The kit can further include a LCN2 biomarker polynucleotide to be used as a positive control. Also included may be enzymes suitable for amplifying nucleic acids including various polymerases (reverse transcriptase, Taq, Sequenase™, DNA ligase etc., depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

In a more specific embodiment, the kit is provided as a PCR kit comprising primers that specifically bind to one or more of the nucleic acid biomarkers described herein. Primers the specifically bind and amplify the target biomarkers described herein include, but are not limited to, LCN2. In specific embodiments, the kit comprises a primer set forth in SEQ ID NO:3 and/or SEQ ID NO:4. The kit can further comprise substrates and other reagents necessary for conducting PCR (e.g., quantitative real-time PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having myocardial injury based on a comparison of the measured levels of one or more biomarkers to one or more reference controls.

The kit can also feature various devices and reagents for performing one of the assays described herein; and/or printed instructions for using the kit to quantify the expression of a LCN2 biomarker gene.

The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

### VI. Fecal Transplant

Fecal microbiota transplantation (FMT), also commonly known as “fecal bacteriotherapy,” represents the one therapeutic protocol that allows the fastest reconstitution of a normal composition and functional gut microbial community. In one aspect, a patient can be treated with a fecal transplant composition.

The FMT compositions of the present invention may be formulated to be compatible with its intended route of administration. A composition of the present invention may be administered by any method suitable for depositing in the gastrointestinal tract, preferably the colon, of a subject. Examples of routes of administration include rectal administration (e.g., by suppository, enema, upper endoscopy, upper push enteroscopy, or colonoscopy), intubation through the nose or the mouth (e.g., by nasogastric tube, nasoenteric tube, or nasal jejunal tube), or oral administration (e.g., by a solid such as a pill, tablet, or capsule, or by liquid).

In particular embodiments, a fecal microbiota preparation comprises a donor's entire or substantially complete microbiota. In one embodiment, a fecal microbiota preparation comprises a non-selective fecal microbiota. In another embodiment, a fecal microbiota preparation comprises an isolated or purified population of live non-pathogenic fecal bacteria.

In a further embodiment, a fecal microbiota preparation comprises a non-selective and substantially complete fecal microbiota preparation from a single donor. In one embodiment, a fecal microbiota preparation described herein comprises a purified or reconstituted fecal bacterial mixture.

In one embodiment, a fecal microbiota transplant composition comprises one or more, two or more, three or more, four or more, five or more, and so forth, isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In another embodiment, a fecal microbiota preparation comprises one or more, one or more, two or more, three or more, four or more, five or more, and so forth, live fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In another embodiment, a fecal microbiota preparation comprises one or more, one or more, two or more, three or more, four or more, five or more, and so forth, live fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In an embodiment, a fecal microbiota in a therapeutic composition comprises a donor's substantially entire or non-selective fecal microbiota, reconstituted fecal material, or synthetic fecal material. In another embodiment, the fecal microbiota in a therapeutic composition comprises no antibiotic resistant population. In another embodiment, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor). Removal of non-living material may be achieved by passing the blended sample through a sieve

In one embodiment, the composition includes at least 2, 3, 4, 5 6 and so forth, different phyla of gut, colon or intestinal bacteria extracted or prepared from the gut, colon or intestine, wherein the phyla include a Bacteroidetes, a Firmicutes, a Proteobacteria a Tenericutes phylum, or a combination thereof, wherein optionally the phyla are chosen from Bacteroidetes, Firmicutes, Proteobacteria, Tenericutes, or a combination thereof, wherein the composition, upon reconstitution with water, includes no greater than about 0.05%, 0.1%, 0.2%, 0.3%, 0.4%, 0.5%, 0.6%, 0.7%, 0.8%, 0.9% 1%, 2%, 3%, 4%, 5%, 6%, 7%, 8%), 9%> or 10%> weight non-living material/weight biological material, wherein the biological material includes human gut, colon or intestinal fecal microbes, and optionally the biological material includes human gut, colon or intestinal bacteria, and wherein optionally the composition includes a pharmaceutically acceptable carrier.

In one embodiment, the composition includes an extract of human feces and a cryoprotectant, wherein the composition, upon reconstitution with water, is substantially odorless, wherein the composition includes biological material, and optionally wherein the biological material includes microbes, and wherein optionally the composition includes a pharmaceutically acceptable carrier, and optionally the composition is a formulation for oral administration.

In one embodiment, the biological material includes: a plurality of prokaryotic cells, eukaryotic cells, or viruses; or a population of prokaryotic cells, eukaryotic cells, and viruses, that is substantially identical to or representative of or equivalent to a population of prokaryotic cells, eukaryotic cells, and viruses present in gut, intestine, colon, or feces of a normal healthy human. In another embodiment, the biological material present includes a population of prokaryotic cells and viruses that is substantially identical to or representative of or equivalent to a population of prokaryotic cells and viruses present in the feces of a normal healthy human. In one embodiment, the biological material includes a population of prokaryotic cells, eukaryotic cells, or viruses that is substantially identical to or representative of or equivalent to a population of prokaryotic cells, eukaryotic cells, and viruses present in the feces of a normal healthy human.

In a further embodiment, the composition includes at least 4 different phyla of bacteria, wherein the phyla include a Bacteroidetes, a Firmicutes, a Proteobacteria, a Tenericutes phyla, or a combination thereof, wherein optionally the phyla are chosen from Bacteroidetes, Firmicutes, Proteobacteria, Tenericutes, or a combination thereof. In one embodiment, the composition further includes at least 5, 6, 7, 8, 9, or 10 different classes of bacteria chosen from Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

Examples of prokaryotic cells that may be present in a fecal microbiota transplant composition include cells that are members of the class Actinobacteria, such as the subclass Actinobacteridae and subclass Coriobacteridae. Examples of the subclass Actinobacteridae include members of the order Actinomycetales, and members of the order Bifidobacteriales. Members of the order Bifidobacteriales include members of the family Bifidobacteriaceae. Examples of the subclass Coriobacteridae include members of the order Coriobacteriales. Members of the order Coriobacteriales include members of the family Coriobacteriaceae.

Other examples of prokaryotic cells include members of the phylum Bacteroidetes, such as class Bacteroidia. Members of class Bacteroidia include order Bacteroidales. Members of order Bacteroidales include members of the family Bacteroidaceae, members of the family Porphyromonadaceae, members of the family Prevotellaceae, and members of the family Rikenellaceae.

Further examples of prokaryotic cells include members of the phylum Firmicutes, such as class Bacilli, Clostridia, Erysipelotrichi, and Negativicutes. Examples of the class Bacilli include members of the order Bacillales (including members of the family Paenibacillaceae and members of the family Planococcaceae) and the order Lactobacillales (including members of the family Aerococcaceae, Enterococcaceae, Lactobacillaceae, and Streptococcaceae). Examples of the class Clostridia include members of the order Clostridiales, and examples of the order Colstridiales include the family Catabacteriaceae, Peptococcaceae, Peptostreptococcaceae, Ruminococcaceae, Clostridiaceae, Eubacteriaceae, and Lachnospiraceae. Examples of the class Erysipelotrichi include members of the family Erysipelotrichaceae. Examples of the class Negativicutes include members of the family Veillonellaceae.

Other examples of prokaryotic cells include members of the phylum Proteobacteria, such as class Alphaproteobacteria, Betaproteobacteria, Deltaproteobacteria, Epsilonproteobacteria, and Gammaproteobacteria. Examples of the class Alphaproteobacteria include members of the order Rhizobiales, and examples of members of the order Rhizobiales includes members of the family Rhodobiaceae, members of the family Brucellaceae, and members of the family Hyphomicrobiaceae. Examples of the class Betaproteobacteria include members of the order Burkholderiales, and examples of members of the order Burkholderiales include members of the family Alcaligenaceae, members of the family Burkholderiaceae, and members of the family Sutterellaceae. Examples of the class Deltaproteobacteria include members of the order Desulfovibrionales, and examples of members of this order include members of the family Desulfovibrionaceae and Desulfomicrobiaceae. Examples of the class Epsilonproteobacteria include members of the order Desulfobacterales, and examples of members of this order include members of the family Desulfobacteraceae. Examples of the class Gammaproteobacteria includes members of the order Alteromonadales and Enterobacteriales. Examples of members of the order Alteromonadales include members of the family Shewanellaceae, and examples of members of the order Enterobacteriales include members of the family Enterobacteriaceae.

Other examples of prokaryotic cells include members of the phylum Tenericutes include members of the class Mollicutes. Examples of the class Mollicutes include members of the order Entomoplasmatales, and members of the order Entomoplasmatales include members of the family Spiroplasmataceae.

Other examples of prokaryotic cells include members of the class Verrucomicrobiae include members of the order Verrucomicrobiales, and examples of members of the order Verrucomicrobiales includes members of the family Verrucomicrobiaceae. Other examples of prokaryotic cells include members of the family Fusobacteriaceae.

### VII. Antibiotic Treatment

In yet another aspect, the present invention provides compositions and methods for treating a patient having SBS. In certain embodiments, the patient is treated with an antibiotic. In particular embodiments, the antibiotic is a broad spectrum antibiotic. In specific embodiments, the treatment comprises a combination of antibiotics.

In certain embodiments, an antibiotic composition comprises an antibiotic selected from the group consisting of rifabutin, clarithromycin, clofazimine, vancomycin, rifampicin, nitroimidazole, chloramphenicol, and a combination thereof. In another embodiment, an antibiotic composition comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

### VIII. LCN2 Inhibitors

In certain embodiments, the LCN2 inhibitor is selected from the group consisting of a small molecule, a polypeptide, a nucleic acid molecule, a peptidomimetic, or a combination thereof. In a specific embodiment, the agent can be a polypeptide. The polypeptide can, for example, comprise an antibody. In another embodiment, the agent can be a nucleic acid molecule. The nucleic acid molecule can, for example, be a LCN2 inhibitory nucleic acid molecule. The LCN2 inhibitory nucleic acid molecule can comprise a short interfering RNA (siRNA) molecule, a microRNA (miRNA) molecule, or an antisense molecule.

A. RNA Interference Agents

In one aspect of the present invention, the expression of LCN2 may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase III promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL. 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-20 (2002).

As used herein, a LCN2 inhibitory nucleic acid sequence can be a siRNA sequence or a miRNA sequence. A 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). An siRNA sequence preferably binds a unique sequence within the LCN2 mRNA with exact complementarity and results in the degradation of the LCN2 mRNA molecule. An siRNA sequence can bind anywhere within the mRNA molecule. An miRNA sequence preferably binds a unique sequence within the LCN2 mRNA with exact or less than exact complementarity and results in the translational repression of the LCN2 mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

As used herein, a LCN2 inhibitory nucleic acid sequence can be an antisense nucleic acid sequence. Antisense nucleic acid sequences can, for example, be transcribed from an expression vector to produce an RNA which is complementary to at least a unique portion of the LCN2 mRNA and/or the endogenous gene which encodes LCN2. Hybridization of an antisense nucleic acid molecule under specific cellular conditions results in inhibition of LCN2 protein expression by inhibiting transcription and/or translation.

The present invention also provides ribozymes as a tool to inhibit LCN2 expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well-known catalytic sequence responsible for mRNA cleavage. See U.S. Pat. No. 5,093,246. Using the known sequence of the target LCN2 mRNA, a restriction enzyme-like ribozyme can be prepared using standard techniques.

B. Small Molecule Inhibitors of LCN2

In one aspect, a LCN2 inhibitor is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Nevertheless, compound libraries may be screened for LCN2 inhibitors. A compound library is a mixture or collection of one or more putative inhibitors generated or obtained in any manner. Any type of molecule that is capable of interacting, binding or has affinity for LCN2 may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative inhibitor or member, i.e., a plurality of members or putative inhibitors. In certain embodiments, a compound library may comprise less than about 50,000, 25,000, 20,000, 15,000, 10000, 5000, 1000, 500 or 100 putative inhibitors, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative inhibitors. In particular embodiments, a compound library may comprise less than about 50,000, 25,000, 20,000, 15,000, 10,000, 5,000, 1000, or 500 putative inhibitors.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.

C. Antibodies to LCN2

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boemer et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

Various procedures known in the art may be used for the production of antibodies to LCN2 or any subunit thereof, or a fragment, derivative, homolog or analog of the protein. Antibodies of the present invention include, but are not limited to, synthetic antibodies, polyclonal antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, e.g., molecules that contain an antigen binding site that immunospecifically binds to an antigen (e.g., one or more complementarity determining regions (CDRs) of an antibody).

Another embodiment for the preparation of antibodies according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics in rational design is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting antibodies disclosed herein, but with altered and even improved characteristics. More specifically, under this rational design approach, peptide mapping may be used to determine “active” antigen recognition residues, and along with molecular modeling and molecular dynamics trajectory analysis, peptide mimic of the antibodies containing antigen contact residues from multiple CDRs may be prepared.

In some embodiments, an antibody specifically binds an epitope of the LCN2 protein. It is to be understood that the peptide regions may not necessarily precisely map one epitope, but may also contain a LCN2 sequence that is not immunogenic. Methods of predicting other potential epitopes to which an immunoglobulin of the invention can bind are well-known to those of skill in the art and include, without limitation, Kyte-Doolittle Analysis (Kyte, J. and Dolittle, R. F., 157 J. MOL. BIOL. 105-32 (1982)); Hopp and Woods Analysis (Hopp, T. P. and Woods, K. R., 78 PROC. NATL. ACAD. SCI. USA 3824-28 (1981); Hopp, T. J. and Woods, K. R., 20 MOL. IMMUNOL. 483-89 (1983); Hopp, T. J., 88 J. IMMUNOL. METHODS 1-18 (1986)); Jameson-Wolf Analysis (Jameson, B. A. and Wolf, H., 4 COMPUT. APPL. BIOSCI. 181-86 (1988)); and Emini Analysis (Emini et al., 140 VIROLOGY 13-20 (1985)).

Amino acid sequence variants of the LCN2 antibodies of the present invention may be prepared by introducing appropriate nucleotide changes into the polynucleotide that encodes the antibody or by peptide synthesis. Such modifications include, for example, deletions from, and/or insertions into and/or substitutions of, residues within the amino acid sequences of the antibody. Any combination of deletions, insertions, and substitutions may be made to arrive at the final construct.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of a polypeptide that increases the serum half-life of the antibody.

Another type of antibody variant is an amino acid substitution variant. These variants have at least one amino acid residue in the antibody molecule replaced by a different residue. For example, the sites of greatest interest for substitutional mutagenesis of antibodies include the hypervariable regions, but framework region (FR) alterations are also contemplated.

A useful method for the identification of certain residues or regions of the LCN2 antibodies that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed antibody variants screened for the desired activity.

Substantial modifications in the biological properties of the antibody can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability, particularly where the antibody is an immunoglobulin fragment such as an Fv fragment.

Another type of substitutional variant involves substituting one or more hypervariable region residues of a parent antibody. Generally, the resulting variant(s), i.e., functional equivalents as defined above, selected for further development will have improved biological properties relative to the parent antibody from which they are generated. A convenient way for generating such substitutional variants is by affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the antibody-antigen complex to identify contact points between the antibody and antigen. Such contact residues and neighboring residues are candidates for substitution according to the techniques elaborated herein. Once generated, the panel of variants is subjected to screening as described herein and antibodies with superior properties in one or more relevant assays may be selected for further development.

It may be desirable to modify the antibodies of the present invention, i.e., create functional equivalents, with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric antibodies with enhanced anti-tumor activity may also be prepared using heterobifunctional cross-linkers as described in Wolff et al., 53 CANCER RESEARCH 2560-65 (1993). Alternatively, an antibody can be engineered which has dual Fc regions and may thereby have enhanced complement lysis and ADCC capabilities.

Stevenson et al., 3 ANTI-CANCER DRUG DESIGN 219-30 (1989).

To increase the serum half-life of an antibody, one may incorporate a salvage receptor binding epitope into the antibody (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the anti-LCN2 antibodies of the present invention.

### IX. AHR Agonists

The abbreviations “AHR” and “AhR” are equivalent and designate both the Aryl hydrocarbon receptor.

The term “AhR agonist” has its general meaning in the art and refers to a compound that selectively activates the AhR. The term “AhR agonist” refers to natural AhR ligands and any compound that can directly or indirectly stimulate the signal transduction cascade related to the AhR. As used herein, the term “selectively activates” refers to a compound that preferentially binds to and activates AhR with a greater affinity and potency, respectively, than its interaction with the other members of bHLH-PAS transcription factors family. Compounds that prefer AhR, but that may also activate other sub-types, as partial or full agonists are contemplated. Typically, an AhR agonist is a small organic molecule or a peptide.

The term “bacterial probiotic” has its general meaning in the art and refers to a useful microorganism that improves the bacterial flora in the gastrointestinal tract and can bring a beneficial action to the host, and a growth-promoting substance therefor. The term “bacterial probiotic” also refers to a bacterium forming the bacterial flora and a substance that promotes the growth of such a bacterium. The term “bacterial probiotic” also refers to a useful microorganism that can bring a beneficial action to a host and substance produced by these microorganisms (microorganism culture). The term “bacterial probiotic” also refers to a dead microbial body and a microbial secretory substance. Because of a suitable enteric environment being formed and the action being independent of differences in enteric environment between individuals, the probiotic is preferably a living microbe.

The term “bacterial probiotic exhibiting AhR activation properties” has its general meaning in the art and relates to a probiotic which can activate the AhR. The term “bacterial probiotic exhibiting AhR activation properties” also relates to a probiotic capable of activating the AhR or having AhR activating potency. The term “AhR activation properties” means potency in being able to activate a signaling pathway that is initiated by AhR activation, and may involve any kind of activating mechanism. Therefore, it is not always necessary for a microbial body itself to be an AhR ligand, and, for example, a secretory substance produced by a microbe may have AhR-activating potency, or the AhR may be activated by a dead microbial body or homogenate thereof. A growth-promoting substance having AhR-activating potency includes a case in which the substance itself has AhR-activating potency and also a case in which the substance itself does not have AhR-activating potency but it promotes growth of a bacterium having AhR-activating potency. Therefore, when a “microorganism” or “bacterium” is referred to or a specific microbe is referred to in the present invention, they include not only a living microbe but also a dead microbial body or homogenate thereof and a culture of said microbe or a secretory substance. However, it is preferably a microbial body itself such as a living microbe or a dead microbial body or homogenate thereof, and from the viewpoint of being capable of forming bacterial flora in the gastrointestinal tract, it is more preferably a living microbe (US 2013/0302844).

In particular embodiments, AHR agonists include dietary indoles, dietary flavonoids, tryptophan metabolites and synthetic weak AHR agonists.

In certain embodiments, an AHR agonist comprises an indolyl derivative (indole derivatives) such as indolyl compounds generated by the tryptophan metabolism and/or derived from dietary intake compounds. Such compounds include, but are not limited to, kynurenine, kynurenic acid, xanthurenic acid, tryptamine (TA), indole acetic acid (IAA); compounds from the serotonin pathway such as hydroxytryptamine, or 5-hydroxytryptamine, 5-Hydroxytryptophan; 6-formylindolo[3,2-b]carbazole (FICZ); metabolites from the commensal bacterial metabolism such as indoxyl sulfate, indole-3-acetic acid (IAA or indole acetate), indole-3-pyruvic acid (I PA, or indole pyruvate), indole-3-carbinol (I3C, or indole carbinol), indole-3-aldehyde (or indole aldehyde), tryptamine, 3-methylindole, indirubin and malassezin.

In more specific embodiments, an AHR agonist from the tryptophan metabolism comprises kynurenine, kynurenic acid, FICZ, IAA, IPA, I3C and idoxyl sulfate. In even more specific embodiments, an AHR agonist from the tryptophan metabolism is FICZ or I3C.

In other embodiments, an AHR agonist comprises benzimidazole derivatives such as omeprazole and lansoprazole. Other examples include primaquine, leflutamide, VAF347 ([4-(3-chloro-phenyl)-pyrimidin-2-yl]-(4trifluoromethyl-phenyl)-amine), TSU-16 ((Z)-3-[(2,4-dimethylpyrrol-5-yl)methylidenyl]-2indolinone), synthetic flavonoids such as TMF (6,2′,4′-trimethoxyflavone) and MNF (3′-methoxy-4′nitroflavone), M50367 (ethyl 3-hydroxy-3-[2-(2-phenylethyl)benzoimidazol-4-yl]propanoate), and M50354 (3-[2-(2-phenylethyl)benzoimidazole-4-yl]-3-hydroxypropanoic acid). See ¶¶ [0036]-[0047] and claims 6-7 of US 2021/0060158.

In certain embodiments, the AHR agonist comprises indoles derivatives, tryptophan catabolites of the microbiota, indole-3-aldehyde (IAld), tryptamine, indole 3-acetate, 3-indoxyl sulfate, 6-formylindolo(3,2-b)carbazole (FICZ), 2,3,7,8-tetrachlorodibenzo-p-dioxin (TCDD), tryptophan derivatives, flavonoids, biphenyls, AhR modulator (SAhRM), diindolylmethane (DIM), methyl-substituted diindolylmethanes, dihalo- and dialkylDIM analogs, mexiletine, polycyclic aromatic hydrocarbon (PAH), polychlorinated biphenyl (PCB), β-naphthoflavone (βNF), 5,6 benzoflavone (5,6 BZF), 3-indoxyl-sulfate (13S),1-(4-Methylphenyl)-2-(4,5,6,7-tetrahydro-2-imino-3(2H)-benzothiazol-yl)ethanone hydrobromide (Pifithrin-α hydrobromide), (2′Z,3′E)-6-Bromo-1-methylindirubin-3′-oxime (MeB10), 5-hydroxy-7-methoxyflavone, 7-methoxyisoflavone, 6-methylflavone, 3-hydroxy-6-methylflavone, pinocembrin (5,7-dihydroxyflavanone) and 7,8,2′-trihydroxyflavone, 1,4-dihydroxy-2-naphthoic acid (DHNA), SU5416, CB7950998 (WO 2012/015914), Nimidipine, Flutamide, Atorvastatin, Leflunomide, Ginseng and natural AhR Agonists (NAhRAs).

AhR agonists also include, but are not limited to, 5-hydroxy-7-methoxyflavone, 7-methoxyisoflavone, 6-methylflavone, 3-hydroxy-6-methylflavone, pinocembrin (5,7-dihydroxyflavanone) and 7,8,2′-trihydroxyflavone.

Other examples of AhR agonists include compound VAF347 [4-(3-chlorophenyl)-N-[4-(trifluoromethyl)phenyl]pyrimidin-2-amine], and its pro-drug version VAG539 [4-(3-chloro-phenyl)-pyrimidin-2-yl]-(4-trifluoromethyl-phenyl)-carbamic acid 2-[(2-hydroxy-ethyl)-methyl-amino]-ethyl ester]. See Lawrence, B. P., 112 BLOOD 1158-65 (2008).

In another embodiment, an AHR agonist comprises Semaxanib (SU5416) [3-(3,5-dimethyl-1H-pyrrol-2-ylmethylene)-1,3-dihydro-indole-2-one]. See Mezrich et al., 7(9) PLoS ONE e44547 (2012).

In a further embodiment, an AhR agonist comprises a selective AhR modulator (SAhRM) such as diindolylmethane (DIM), methyl-substituted diindolylmethanes, dihalo- and dialkylDIM analogs, mexiletine, 0-naphthoflavone (3NF) (5,6 benzoflavone (5,6 BZF) and moieties described, for example, in Safe et al., 135 TOXICOL SCI. 1-16 (2013); Furumatsu et al., 56 DIG DIS SCI 2532-44 (2011); and WO 2012/015914.

In particular embodiments, an AHR agonist comprises a bacterial probiotic. Bacterial probiotics include, but are not limited to, bacterium exhibiting naturally AhR activation properties or modified bacterium exhibiting AhR activation properties such as Allobaculum, Lactobacillus reuteri, Lactobacillus taiwanensis, Lactobacillus johnsonii, Lactobacillus animalis, Lactobacillus murinus, the genus Adlercreutzia, the phylum Actinobacteria, lactic acid bacterium, Lactobacillus bulgaricus, Streptococcus thermophilus, Bifidobacterium, Propionic acid bacterium, Bacteroides, Eubacterium, anaerobic Streptococcus, Enterococcus, Lactobacillus delbrueckii subsp. Bulgaricus, Escherichia coli, other intestinal microorganisms and probiotics. See US 2013/0302844. See also ¶¶s [0074]-[0081] and claims 19-25 of US 2019/0282638.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: A Central Role for Lipocalin-2 in the Adaptation to Short-Bowel Syndrome Through Down-Regulation of IL22 in Mice

Changes in the intestinal microbiota have been shown to lead to changes in intestinal resistance,⁷ demonstrating the interplay between the two. Recently, Helmrath et al. noted that the microbiota is a key mediator of gut homeostasis and a potential driver of metabolism and immunomodulation after intestinal loss.¹¹ LCN2 is a glycoprotein expressed in neutrophils that is involved in the antibacterial innate immune response.¹² LCN2 binds to siderophores, which are bacterial peptides that bind iron, and thus prevents bacterial iron uptake and acts as a bacteriostatic agent.^{13, 14} Moreover, LCN2^{-/-} mice have been shown to have decreased survival following Proteobacteria (E. coli) infection compared to wild-type (WT) control mice.¹⁵ The present inventors have previously shown that Lcn2 mRNA expression increased following cholestasis in a cholestatic mouse model,¹⁶ and Teitelbaum et al. showed upregulation of Lcn2 gene expression in mice that underwent a 70% small bowel resection (SBR) one week earlier;¹⁷ however, a link between LCN2 and SBS remained to be established. These findings led us to hypothesize that increase of LCN2 expression following SBS may lead to changes in the microbiota and improve intestinal adaptation. To test this hypothesis, we chose the 75% SBR mouse model originally described by Helmrath et al.,¹⁸ because it recreated the malabsorption seen in SBS and retained the powerful advantage of easy genetic manipulations to study mechanisms involved in the host-gut microbiota crosstalk in intestinal adaptation.

As described herein, LCN2 expression increased following SBS; however contrary to our hypothesis, this increased LCN2 expression led to increased inflammation and a detrimental effect on intestinal adaptation through an increased intestinal dysbiosis and IL-22 inhibition. Furthermore, rescue therapy with exogenous IL-22 improved adaptation and counteracted the dysbiosis seen in our SBS model. These findings supported that LCN2 inhibition or restoration of IL-22 as potential therapeutic targets to augment intestinal adaptation in SBS.

### Materials and Methods

Animals and Housing. Under the Johns Hopkins University Animal Care and Use Committee-approved protocol #MO18M194, we performed a 75% small bowel resection (SBR) and sham operation on either C57BL/6J, LCN2^{-/-} or IL-22^{-/-} mice. C57BL6/J WT and LCN2^{-/-} mice were obtained from Jackson Laboratories (Bar Harbor, Me.). IL-22^{-/-} mice were obtained from Genentech, Inc. Conventional mice were bred and housed at Johns Hopkins University Animal Facility with a 12-hour light-dark cycle and given rodent chow ad libitum after weaning. At 5 weeks of age, male conventional mice were individually housed and fed with rodent liquid diet (Liber-DeCarli '82, Bio-serv, F1259SP) for four days before surgery. Rodent liquid diet was maintained during post-operative period. Fecal microbiota transplant (FMT) was performed in germ-free (GF) mice (C57BL/6J background), which were bred and housed in germ-free mouse core at Johns Hopkins University.

Creation of a clinically relevant model of short bowel syndrome in the mouse. In our laboratory, we established the model previously described by Helmrath et al.¹⁸ Briefly, the hair on the abdomen of the mouse was removed by depilatory creams, and the abdomen was scrubbed with three rounds of betadine, prior to making an incision. Through a 1.5 cm midline laparotomy, the small bowel was examined. The 75% SBR was performed by transecting 3 cm distal to the ligament of Treitz and 6 cm proximal to the cecum. The intervening proximal small bowel (approximately 18 cms) was removed. Intestinal continuity was restored with a primary end-to-end jejunoileal anastomosis using 8-0 Nylon suture. The sham operation (SHA) was performed by transecting the small bowel 6 cm proximal to the cecum and immediately creating a primary end-to-end ileoileal anastomosis. Subcutaneous closure was performed with a running absorbable 4-0 Vicryl suture. Cutaneous closure was completed by interrupted 4-0 silk sutures. All animals received 1 mL of saline subcutaneously before and immediately after procedure.

After surgery, mice were offered free access to water for the first 24 hours. Fresh rodent liquid diet was provided and body weight was measured daily. Bromodeoxyuridine (BrdU) (Invitrogen, 00-0103) was administered through intraperitoneal injection (ip) 24 hours before euthanasia. Intestinal permeability was evaluated with FITC-Dextran intestinal permeability assay as described below. Serum, intestinal issue, liver, and cecal content were collected after euthanasia.

FITC-Dextran Intestinal Permeability Assay. Mice were fasted for 2 hours. Serum was collected prior to the FITC-Dextran administration as blank. Then, the mice were gavage fed FITC-Dextran (44 mg/100 g, Sigma, FD4). Serum was collected again 1 hour afterwards in conventional mice and 4 hours afterwards in germ-free mice. Fluorescence was measured on samples diluted with PBS (1:2) using an excitation wavelength of 490 nm and an emission wavelength of 520 nm. Fold changes of FITC-Dextran in the serum 1 hour/blank or 4 hours/blank were calculated.

Depletion of Murine Intestinal Microbiota. Four-week old WT C57BL/6J male mice were administered an antibiotic cocktail [vancomycin hydrochloride (0.5 g/L, Gold Biotechnology, V-200, USP, St. Louis, Mo., USA), metronidazole (1 g/L, Gold Biotechnology, M-840, USP, St. Louis, Mo., USA), neomycin Sulfate (1 g/L, Gold Biotechnology N-620, USP, St. Louis, Mo., USA), ampicillin sodium (1 g/L, Gold Biotechnology, A-301, USP, St. Louis, Mo., USA)] in the rodent liquid diet (Lieber-DeCarli '82, BioServe F1259SP, Flemington, N.J., USA) for two weeks before small bowel resection. The liquid diet and antibiotic cocktail were made fresh and refilled daily in the feeding tube. The control group was administered rodent liquid diet only. Mouse fecal pellets were collected on days 0, 4, and 7 during antibiotic cocktail treatment. Fecal DNA were isolated with Dneasy PowerSoil kit (QIAGEN, 12888-100, Carlsbad, Calif., USA) according to the manufacturer's instruction. Microbiota depletion was confirmed by fecal total bacterial load via 16S rRNA PCR assay in both the antibiotic cocktail (ABX) treatment group and the control group. 16S rRNA universal primers were: 5′-ACTCCTACGGGAGGCAGCAGT-3′ (SEQ ID NO:1) and 5% ATTACCGCGGCTGCTGGC-3′ SEQ ID NO:2). Survival rate, body weight, length of villi in jejunum, and LCN2 protein expression in the jejunum and liver were assessed by western blot.

Assessment of LCN2, IL-6, and IL-22. LCN2, IL-6, and IL-22 in the serum or feces were quantified with ELISA kits following instruction. Measurements were carried out using Mouse Lipocalin2 DuoSet (R&D, DY1857-05), Mouse IL-6 DuoSet (R&D. DY406-05), IL-22 Mouse ELISA Kit (Invitrogen, 88-7422-22). LCN2 expression in intestine and liver were measured by western blot analysis. Primary antibodies goat anti-lipocalin2 (1:500, R&D, AF1857, Minneapolis, Minn., USA) were incubated with PVDF membrane at 4° C. overnight. Secondary antibodies anti-goat IgG (H+L) (1:3000, R&D, HAF109, Minneapolis, Minn., USA) were incubated with PVDF membrane for 1 hr. at RT.

Morphometric Analysis. The jejunum and ileum were fixed in 10% neutral buffered formalin for 24 hours. Fixed tissues were embedded in paraffin and cut in 5 μm sections for hematoxylin and eosin (H&E). Villus length in jejunum and ileum was measured under light microscopy (EVOS FL Auto Imaging System). At least 30 well-oriented intact villi were counted per mouse.

Immunohistofluorescence. The intestine tissues were fixed in 4% paraformaldehyde (PFA) for 24 hours. Fixed tissues were embedded in paraffin and cut in 5 μm sections for immunofluorescence staining. Slides were subjected to a deparaffinization and rehydration process. Antigen retrieval was performed in 10 mM Citric Acid buffer pH 6.0 for 10 min at 100° C. Slides were then washed with PBS and blocked with 1% BSA (Cell Signaling Technology, 9998S, Danvers, Mass., USA) and 5% donkey serum (Sigma, D9663, St. Louis, Mo., USA) at RT for 1 h. Primary antibody goat anti-lipocalin 2 (1:100, R&D, AF1857, Minneapolis, Minn.), rabbit anti-myeloperoxidase (MPO) (1:400, Thermo Scientific, RB-373-A, Fremont, Calif., USA), rat anti-F4/80 (1:200, Invitrogen, 14-4801-82, Carlsbad, Calif., USA), rat anti-BrdU (1:300, Novus, NB500-169, Centennial, Colo., USA) was applied on tissue at 4° C. overnight. Tissues were incubated with secondary antibody donkey anti-goat IgG (H+L) Alexa Fluor 555 (1:1000, Invitrogen, A-21432, Carlsbad, Calif., USA), donkey anti-rabbit IgG (H+L) Alexa Fluor 680 (1:500, Invitrogen, A10043, Carlsbad, Calif., USA), donkey anti-rat IgG (H+L) Alexa Fluor 488 (1:500, Invitrogen, A-21208, Carlsbad, Calif., USA) at RT for 2 hours. Slides were rinsed with PBS, counterstained and mounted in VECTASHIELD with DAPI (Vector Laboratories, H-1200, Burlingame, Calif., USA). Imaging was performed with a confocal microscope (Nikon Eclipse Ti).

Fecal DNA Isolation and 16S rRNA Gene Sequencing. Fecal DNA were isolated from cecal content with DNeasy PowerSoil kit (Qiagen, 12888-100). V3-V4 regions of the 16S rRNA gene sequencing was performed on the Illumina MiSeq platform at the Johns Hopkins Transcriptomics and Deep Sequencing Core. Subsequently, intestinal microbiome was analyzed by Resphera Biosciences.¹⁹⁻²¹ Raw paired-end reads output by the MiSeq platform was merged into consensus fragments by FLASH and subsequently filtered for quality/length using Trimmomatic/QIIME. Passing sequences were trimmed of primers, evaluated for chimeras with UCLUST, and screened for mouse-associated contaminant using Bowtie2. Chloroplast/mitochondrial contaminants were detected and filtered using the RDP classifier. High-quality 16S sequences were assigned to OTUs with a high-resolution taxonomic lineage using Resphera Insight. Contaminants were identified by searching for highly enriched species/OTUs in available, negative control blank tubes (minimum 10-fold enrichment in blanks compared to non-blanks). Contaminant-filtered sequences were further analyzed by PICRUSt to infer functional content and aggregated at KEGG levels 1, 2, or 3. Taxonomic profiles were then subsampled to an even level of coverage prior to downstream statistical comparisons. Alpha and beta-diversity analyses was performed using QIIME. PERMANOVA was applied for beta-diversity comparative analysis. Differential abundance analysis was performed for all taxonomic groups (phylum through species/OTUs), alpha diversity measures and PICRUSt functional categories. P-values were adjusted using the False Discovery Rate (FDR) to account for multiple hypothesis testing.

Creation of Fecal Slurry from Cecal Contents and Fecal Microbiota Transplant (FMT). 5-weeks old GF mice were randomly divided into two groups. Body weight was obtained before FMT. The 4% fecal slurry were created from cecal contents of conventional WT SBR and LCN2^{-/-} SBR mice respectively and were gavage fed (100 μL/mouse) to GF mice. 7 days after FMT, body weight, intestinal permeability, and histology was evaluated.

Intestinal Laminal Propria Lymphocytes Isolation and Cell Surface and Intracellular Staining for Flow Cytometry. Small intestine was excised and mesenteric fat tissue along the intestine was removed. The small intestine was opened longitudinally and cut into 5 mm pieces. After incubation with 0.1 mM ethylenediaminetetraacetic acid (EDTA) in Dulbecco's Modified Eagle Medium (DMEM) (Gibco, 21068-028, Carlsbad, Calif., USA) with 10% fetal bovine serum (FBS) (Corning, 35-010-CV, N.Y., USA) for 1 h at 37° C. 200 rpm, epithelial cells and intraepithelial lymphocytes (IELs) were removed by passage through a 100 μM cell strainer. The remaining lamina propria, muscle and serosa layers were mechanically minced and incubated with DMEM with 50 unit/mL collagenase (Worthington-Biochemical Corporation, LS004130, Lakewood, N.J. USA) for 45 minutes at 37° C. 200 rpm. A single cell suspension was obtained by passing the cells through a 70 μM cell strainer, and then subjecting the cells to a Percoll gradient (44% and 67%) (Sigma, GE17-0891-01, St. Louis, Mo., USA) separation at room temperature (RT) for 20 minutes at 600 g. Lamina propria lymphocytes (LPLs) were collected from the interface between 44% and 67% Percoll solution and washed with PBS.

LPLs were stimulated in complete media (RPMI 1640 with 2 mM L-glutamine, 10% FBS, 50 mM β-ME, 100 U/ml penicillin-streptomycin) with cell stimulation cocktail plus protein transport inhibitors (eBioscience, 00-4975-03, Carlsbad, Calif., USA) which were formulated with phorbol 12-myristate 13-acetate (PMA)(80 nM), ionomycin (1.34 μM), brefeldin A (10.6 μM) and monensin (2 μM) at 37° C. for 5 hours. LPLs were then fixed and permeabilized (BD, 554714, Franklin Lakes, N.J., USA) at 4° C. for 20 minutes and stained with an antibody cocktail, including PerCP-Cy5.5 rat anti-mouse CD4 (clone RM4-5, 1:200, BD, 561115), and APC rat anti-mouse/human/monkey IL-22 (IL22JOP, 1:100, eBioscience, 17-7222-80, Carlsbad, Calif., USA) in 1×Perm/Wash buffer (BD, 554723, Franklin Lakes, N.J., USA) at 4° C. for 30 minutes. The cells were washed twice with 1× Perm/Wash buffer and suspended in FACS buffer (PBS with 0.5% BSA, Sigma, A8412, St Louis, Mo., USA) before flow-cytometric analysis.

Naïve CD4⁺ T Lymphocytes Isolation and Th22 cells Differentiation. We isolated naïve CD4+ T cells as previously described by Bedoya S K et al.²² Briefly, splenic tissue was removed from 6-week old C57BL/6J male mice and ground into a single cell suspension under a sterile hood. Red blood cells (RBC) were removed by using ammonium-chloride-potassium (ACK) lysing buffer (Quality Biological, 118-156-101, Gaithersburg, Md., USA) at RT for 3 minutes. Naïve CD4+ T cells were isolated from total splenocyte by magnetic activated cell sorting (MACS) based on positive selection using CD4 (L3T4) MicroBeads (Miltenyi Biotec, 130-117-043, Bergisch-Gladbach, Germany) according to the manufacturer's instructions. The purity of naive CD4+ T cells was more than 97%, as measured by flow cytometry.

For Th22 cell differentiation, CD4+ T cells were stimulated in complete media (RPMI 1640 with 2 mM L-glutamine, 10% FBS, 50 mM 1-ME, 100 U/ml penicillin-streptomycin) with plate-bound anti-CD3ε (Clone 145-2C11, 1 ug/mL, BD; 550275) and soluble anti-CD28 (Clone 37.51, 2 ug/ml, BD, 553295) for 5 days under polarizing condition for Th22 cells (IL-6 20 ng/mL, IL-β 10 ng/mL, TNF-α 10 ng/mL)²³ with and without mouse recombinant (rm) LCN2 (1 ug/mL, R&D, 1857-LC-050). Cell supernatant was collected for IL-22 ELISA analysis (Invitrogen, 88-7422-22) according to the manufacturer's instructions.

Rescue IL-22 Therapy for SBR WT Mice. Following a 75% SBR, WT mice were treated daily for six days via intraperitoneal injections with either 100 μL PBS as control or 100 μL PBS containing 4 μg rmIL-22 (PeproTECH, 210-22) as treatment. At post-operative day (POD) 7, intestinal tissue and cecal contents were collected after euthanasia. The relative abundance of Proteobacteria, Bacteroidetes, Firmicutes, Lactobacillus and Enterobacteriaceae of fecal microbiome and Reg3b and Reg3g genes in the intestine were assessed by q-PCR.

RNA Extraction and Reverse Transcription. Total RNA from tissue was extracted according to the manufacturer's instructions (QIAGEN, RNeasy Mini Kit, 74106). Genomic DNA was eliminated from RNA, and total RNA (0.5 μg) was reverse transcribed to cDNA with QuantiTect Reverse Transcription Kit according to the manufacturer's protocol (QIAGEN, 205311, Germantown, Md., USA).

Quantitative real-time PCR. Gene quantification was carried out with iTap Universal SYBR Green Supermix (Bio-rad, 172-5124, Hercules, Calif., USA) on a CFX96 Touch Real-time PCR system (Bio-rad) according to the manufacturer's instructions and calculated with 2^{-ΔΔCT} method. Thermal cycling was performed at 95° C. for 5 min, followed by 40 cycles at 95° C. for 15 s, 60° C. 10s, 72° C. for 30 s. Primers for transcriptional levels of Lcn2, IL-6. Sucrase-isomaltase, Tnf-α, IL-22, Reg3b, and Reg3g in intestine or liver are listed in Table 1. Primers for fecal microbiome analysis in WT SBR mice which treated with rmIL-22mRNA are listed in Table 2.

TABLE 1
Primers

| | | | Amplicon |
|---|---|---|---|
| | | | length |
| Gene | Forward primer | Reverse primer | (bp) |
| Len2 | AAGGAACGTTTCACCCGCTT (SEQ | AATGCATTGGTCGGTGGGGA (SEQ | 84 |
| | ID NO: 3) | ID NO: 4) | |
| Tnf-α | TTCCGAATTCACTGGAGCCTCGAA | TGCACCTCAGGGAAGAATCTGGAA | 144 |
| | (SEQ ID NO: 5) | (SEQ ID NO: 6) | |
| IL-6 | CCAATTTCCAATGCTCTCCT (SEQ | ACCACAGTGAGGAATGTCCA (SEQ | 182 |
| | ID NO: 7) | ID NO: 8) | |
| IL-22 | CGACCAGAACATCCAGAAGAA | GAGACATAAACAGCAGGTCCA (SEQ | 110 |
| | (SEQ ID NO: 9) | ID NO: 10) | |
| Reg3g | GTACCCTGTCAAGAGCCTCA (SEQ | GTACCCTGTCAAGAGCCTCA (SEQ | 184 |
| | ID NO: 11) | ID NO: 12) | |
| Reg3b | GCTCAATAGCGCTGAGGCTT (SEQ | AGAAAGCACGGTCTAAGGCA (SEQ | 200 |
| | ID NO: 13) | ID NO: 14) | |
| Hprt | GCTGACCTGCTGGATTACATTAA | TGATCATTACAGTAGCTCTTCAGTCT | 101 |
| | (SEQ ID NO: 15) | GA (SEQ ID NO: 16) | |
| Sucrase- | ATCCAGGTTCGAAGGAGAAGCAC | TTCGCTTGAATGCTGTGTGTTCCG | 154 |
| isomaltase | T (SEQ ID NO: 17) | (SEQ ID NO: 18) | |
| Tgr5 | CAGCTGCCCAAAGGTGTCTA (SEQ | CAAGTCCAGGTCAATGCTGC (SEQ | 110 |
| | ID NO: 19) | ID NO: 20) | |

TABLE 2
16S rRNA gene target group primers

| | | | Amplicon |
|---|---|---|---|
| Target group | Forward primer | Reverse primer | length (bp) |
| Deltaproteo- | GCTAACGCATTAAGTRYCCCG | GCCATGCRGCACCTGTCT | 189 |
| bacteria | (SEQ ID NO: 21) | (SEQ ID NO: 22) | |
| Betaproteo- | AACGCGAAAAACCTTACCTACC | TGCCCTTTCGTAGCAACTAGT | 174 |
| bacteria | (SEQ ID NO: 23) | G (SEQ ID NO: 24) | |
| Epsilonproteo- | TAGGCTTGACATTGATAGAATC | CTTACGAAGGCAGTCTCCTTA | 189 |
| bacteria | (SEQ ID NO: 25) | (SEQ ID NO: 26) | |
| Bacteroidetes | GGARCATGTGGTTTAATTCGATG | AGCTGACGACAACCATGCAG | 127 |
| | AT (SEQ ID NO: 27) | (SEQ ID NO: 28) | |
| Firmicutes | GGAGYATGTGGTTTAATTCGAA | AGCTGACGACAACCATGCAG | 123 |
| | GCA (SEQ ID NO: 29) | (SEQ ID NO: 30) | |
| Universal 16S | AAACTCAAAKGAATTGACGG | CTCACRRCACGAGCTGAC | 136 |
| | (SEQ ID NO: 31) | (SEQ ID NO: 32) | |

Statistical Analysis. Statistical analysis was performed using Prism 5.0 (GraphPad Software). Student's t-test was used to analyze statistical differences between two groups. ANOVA with a Bonferroni post-test correction was used to calculate statistical differences between 3 or 4 groups. 16S rRNA microbiome analysis was described in Supplemental Methods. Values of P<0.05 were considered significant.

### Results

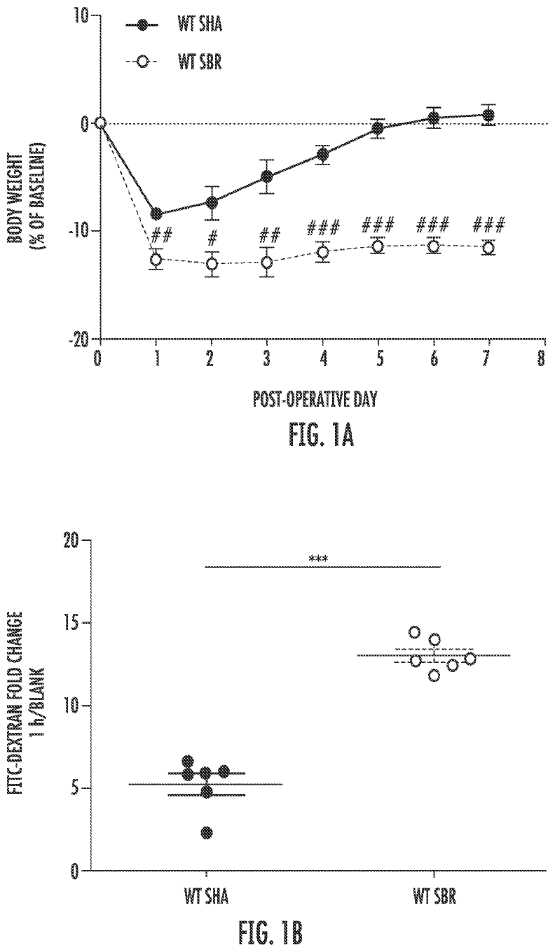

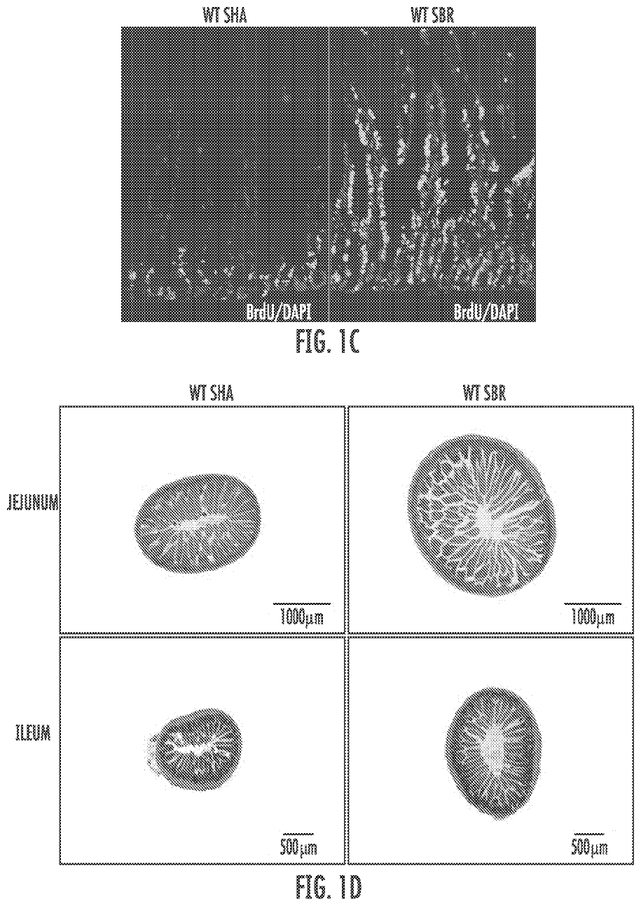

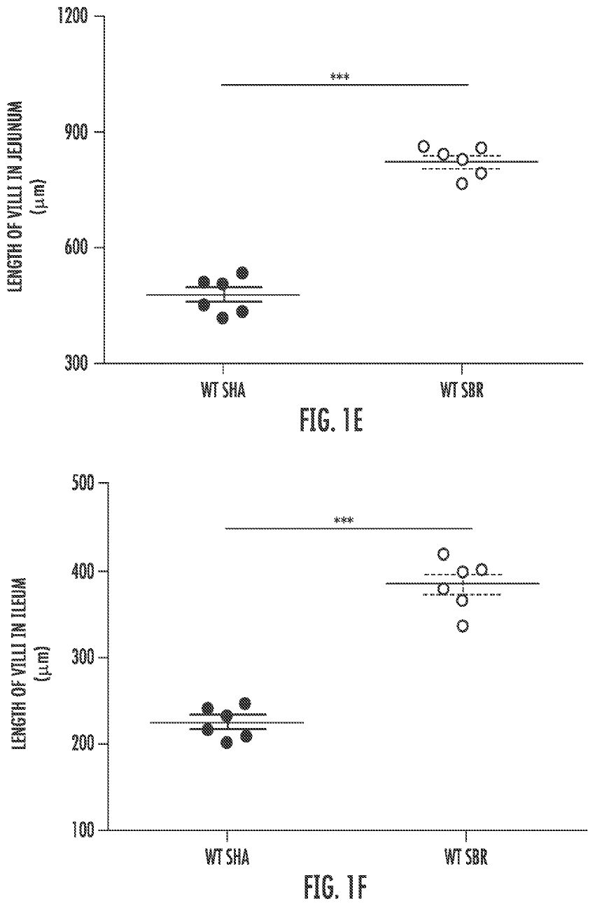

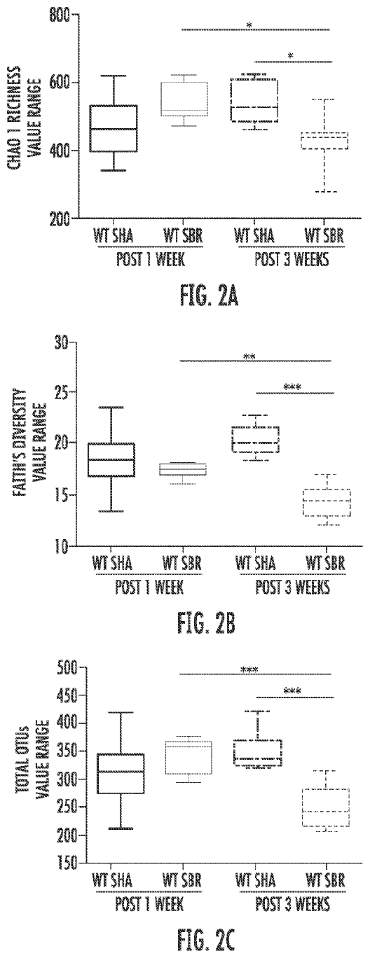

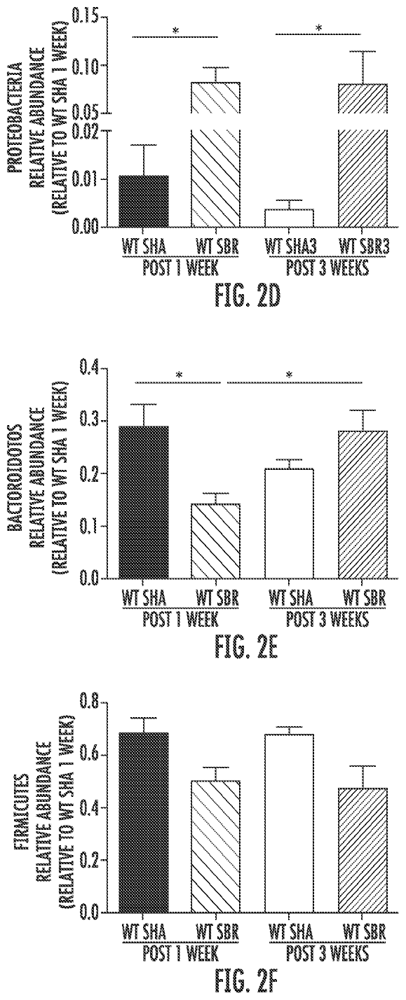

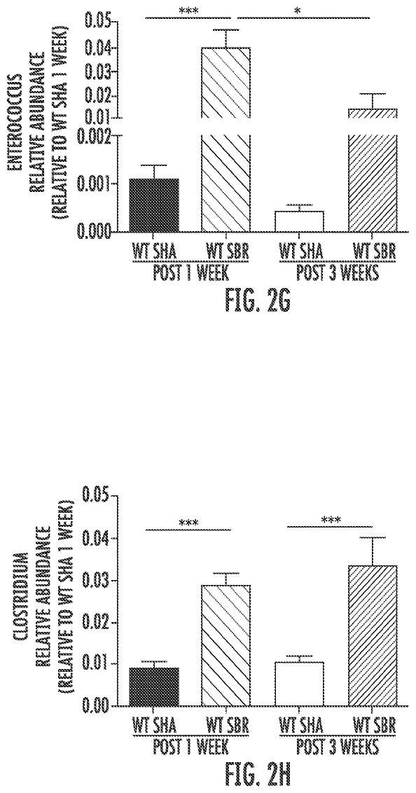

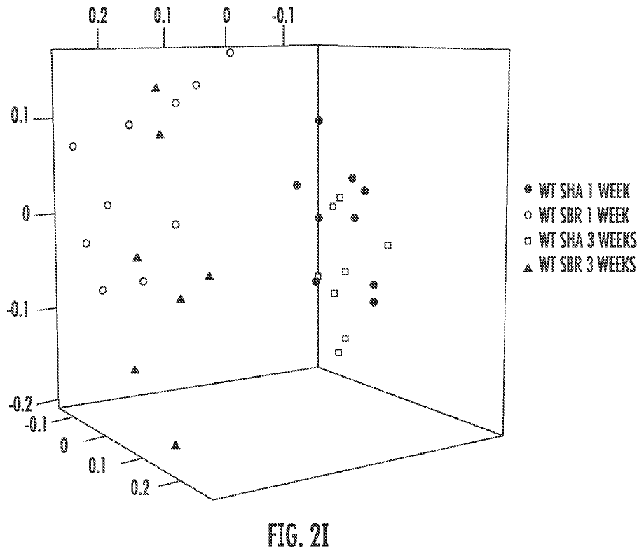

75% SBR in a Mouse Recreated Increased Intestinal Permeability, Enterocyte Proliferation and Intestinal Dysbiosis Seen in SBS. Mice entering the current model of SBS lost 10⁻¹⁵% of their body weight by POD 7; whereas, the sham-operated (SHA) mice started to gain weight on POD2 and recovered quickly to baseline by POD 7 (FIG. 1A). Intestinal adaptation and enterocytes proliferation were observed in the WT SBR mice as evidenced by increased BrdU positive enterocytes along the intestinal villi (FIG. 1C), as well as increased length of villi in jejunum and ileum (FIG. 1D-1F). FITC-Dextran in the serum significantly increased following gavage feeding in mice underwent 75% SBR, suggesting high intestinal permeability in SBS (FIG. 1B). Chao 1 richness (FIG. 2A), Faith' diversity (FIG. 2B), and total OTUs (FIG. 2C) were used to identify within individual taxa richness and evenness. In the long term, there was significant decreased alpha diversities in the WVT SBR mice as compared to the WT SHA mice at post-operative 3 weeks. The principal component analysis (PCA) results showed that intestinal microbiome profile was clearly divided into two groups. SHA and SBR at post-operative 1 week and 3 weeks (FIG. 2I). There were significantly increased relative abundance of Proteobacteria, Enterococcus, and Clostridium and decreased of Bacteroidetes and Firmicutes in the SBS mice (FIG. 2D-2H). These changes in the microbiota prompted us to evaluate the antibacterial peptide, LCN2 in the host response to SBS.

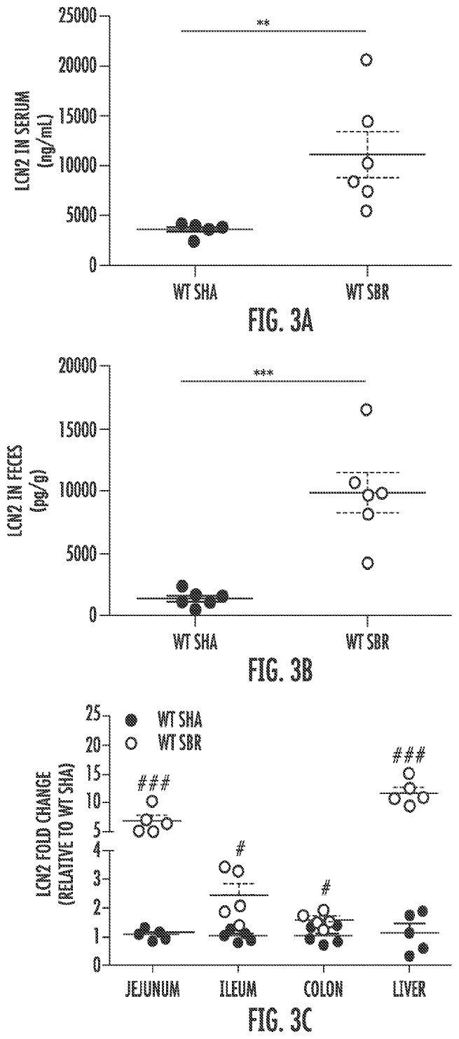

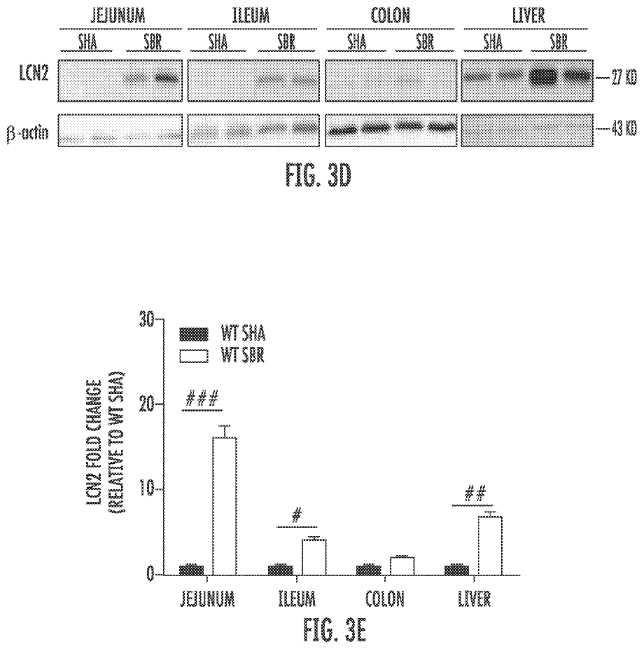

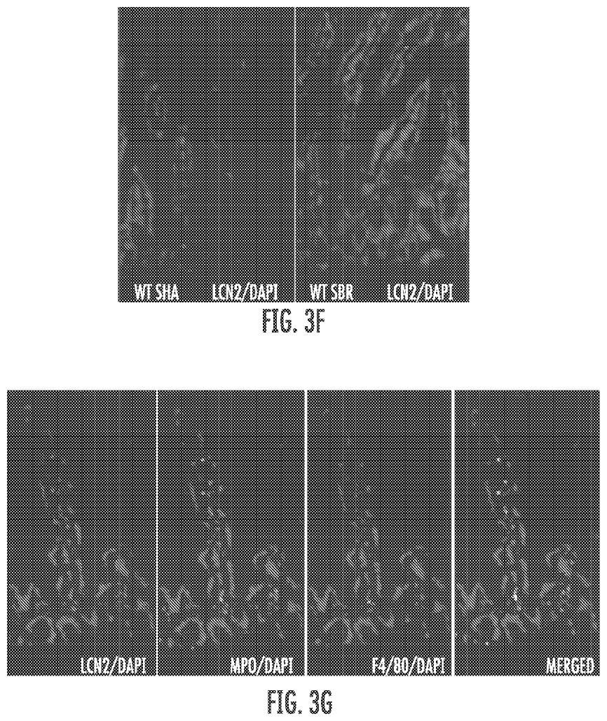

LCN2 Expression Increased Following 75% SBR. At the onset of our analysis, there was significant increase of LCN2 protein in the serum and feces after 75% SBR at POD 7 (FIGS. 3A-3B) Compared to SHA counterparts, transcriptional levels of Lcn2 gene and LCN2 protein significantly increased following 75% SBR, especially in the jejunum and the liver (FIGS. 3C-3E). Immunofluorescence staining of jejunum from SHA and SBR mice showed that LCN2 positive cells localized at lamina propria (FIG. 3F). Most of LCN2 co-localized with neutrophil marker myeloperoxidase (MPO) (FIG. 3G).

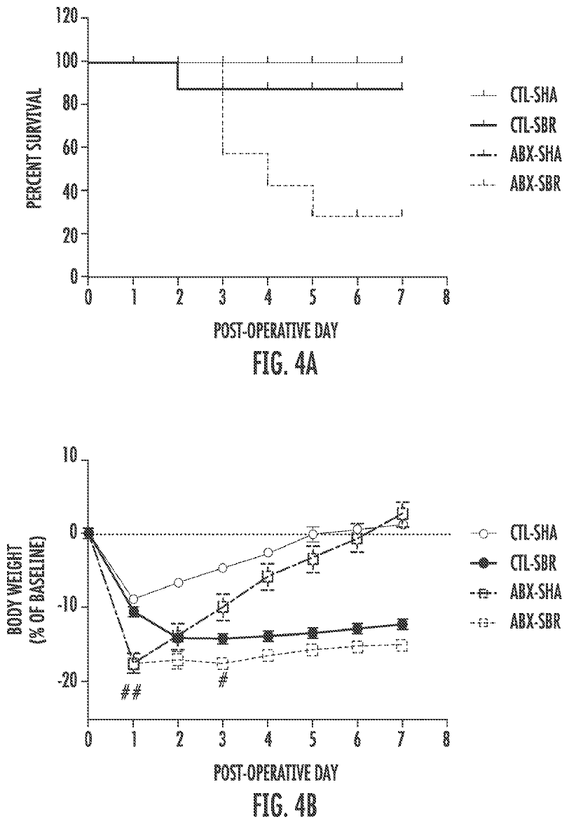

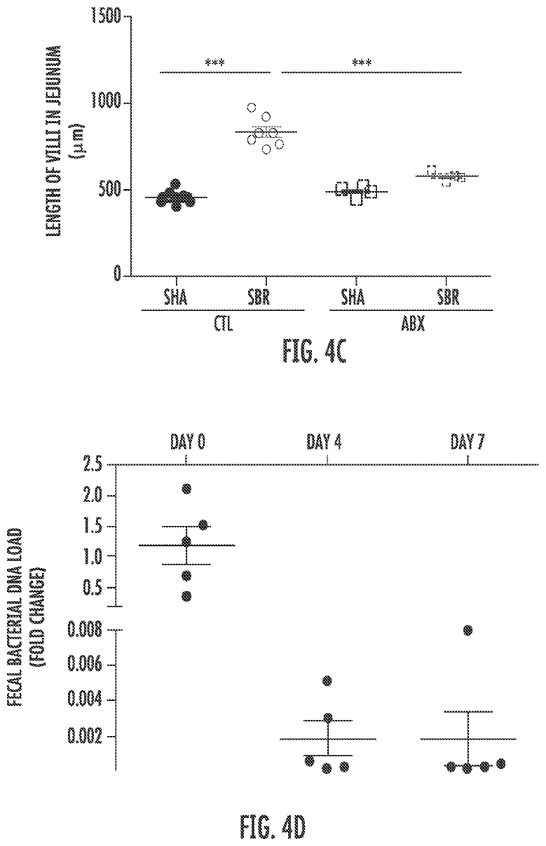

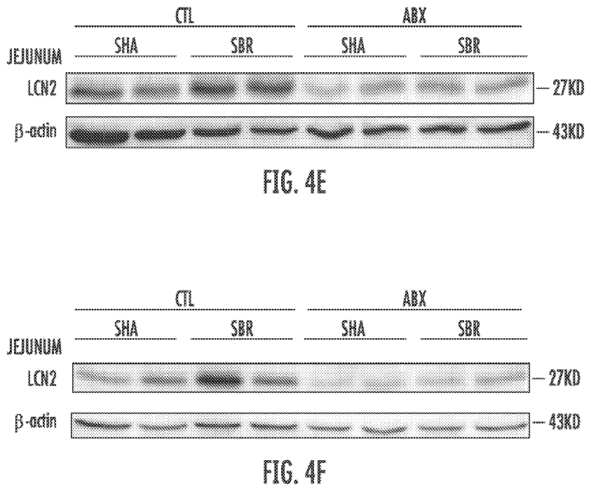

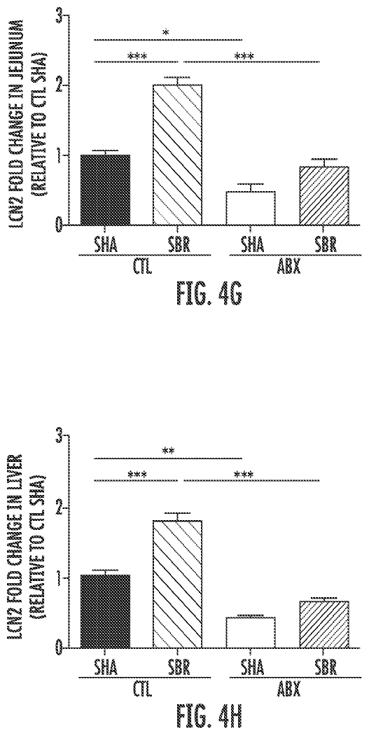

Depletion of Intestinal Microbiota Decreased Intestinal Adaptation in SBS. Antibiotics (ABX) treatment successfully deleted intestinal microbiota within 7 days in WT mice, which was evidenced by significant decrease of fecal bacterial load (FIG. 4C). However, depletion of intestinal microbiota significantly decreased survival rate (FIG. 4A) and intestinal adaptation in SBR mice with ABX treatment, which lost more body weight (FIG. 4B), had shorter villi in jejunum (FIG. 4C) comparing to SBR mice with intestinal microbiota. We also found that ABX treatment abolished LCN2 expression in the intestine and liver (FIG. 4E-4H).

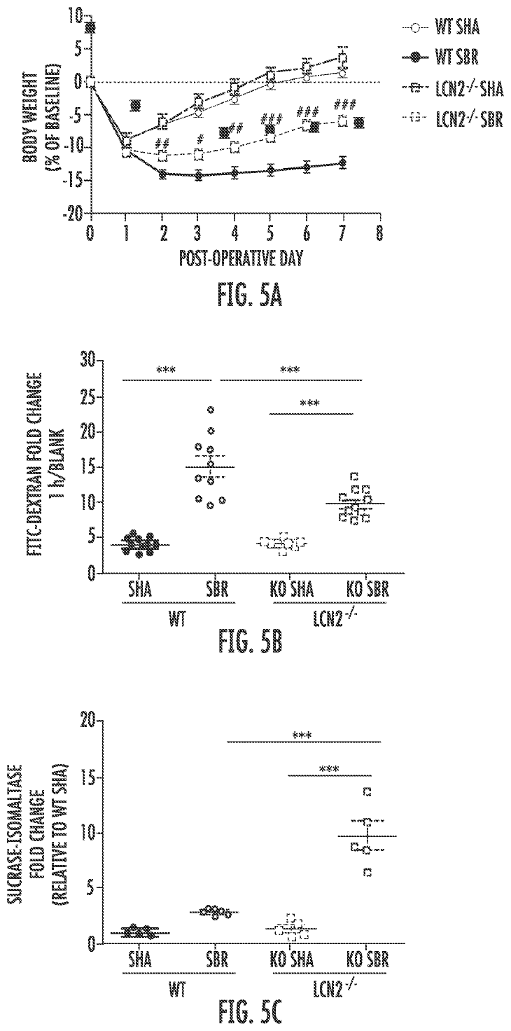

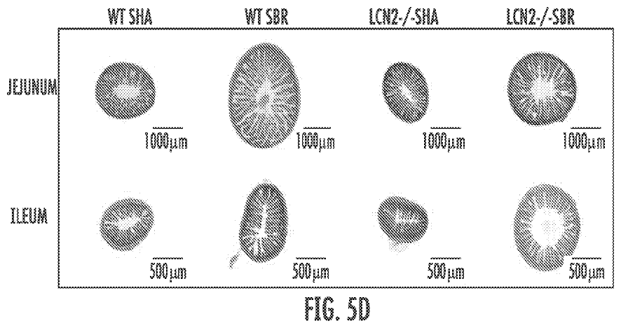

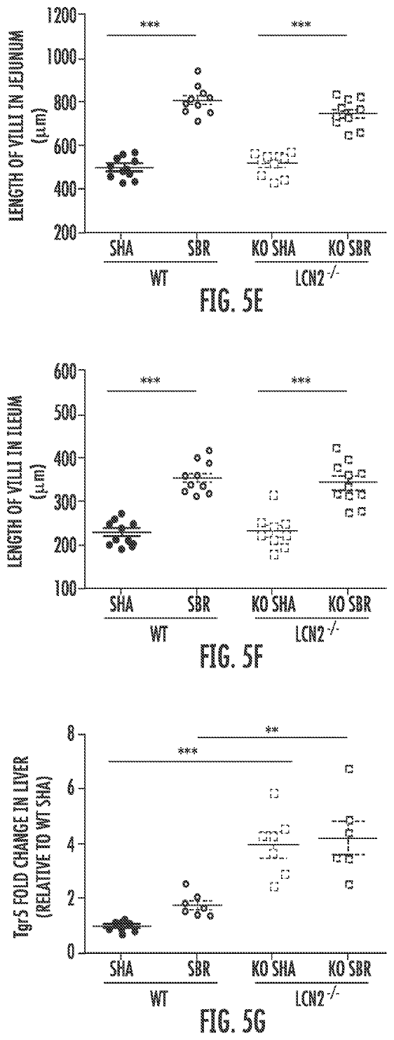

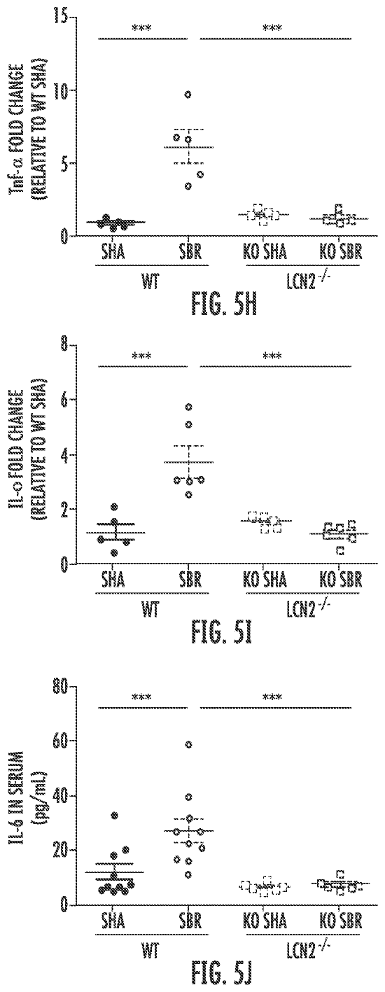

LCN2^{-/-} Mice had less Intestinal Inflammation and Greater Adaptation as evidenced by less Intestinal Permeability, Increased Carbohydrate Enzyme Expression, less Weight Loss, Less Dysbiosis and Greater Survival Following 75% SBR than WT Mice. We hypothesized that LCN2 was beneficial to the host response to SBS and that the absence of LCN2 would be detrimental. To test our hypothesis, we subjected LCN2^{-/-} mice to our SBS model. Contrary to our hypothesis, we noted that the LCN2^{-/-} SBR mice lost significantly less body weight (FIG. 5A) and had lower intestinal permeability (FIG. 5B) than WT SBR mice. The length of the villi in the jejunum and ileum significantly increased after SBR in both WT and LCN2^{-/-} mice (FIG. 5D-F), we didn't see significant difference in proliferation between two genotypes. There were significant increase of sucrase-isomaltase in the intestine (FIG. 5C) and Tgr5 in the liver (FIG. 5G) of LCN2^{-/-} SBR mice, but not in WT SBR mice. Moreover, LCN2^{-/-} SBR mice had less regional and systemic inflammation than WT SBR mice, which were showed that significantly lower transcriptional levels of IL-6 and Tnf-α genes in the intestine and lower IL-6 expression level in serum (FIG. 5H-5J).

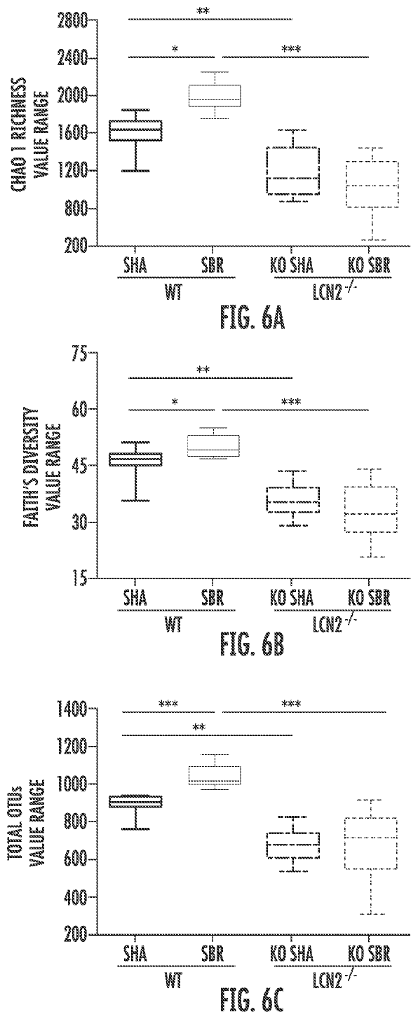

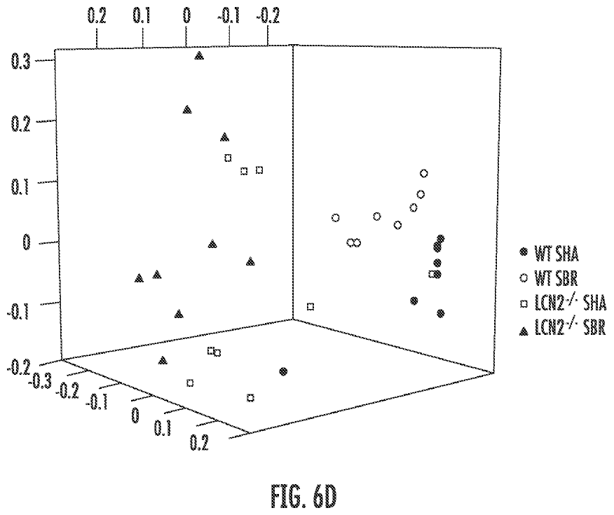

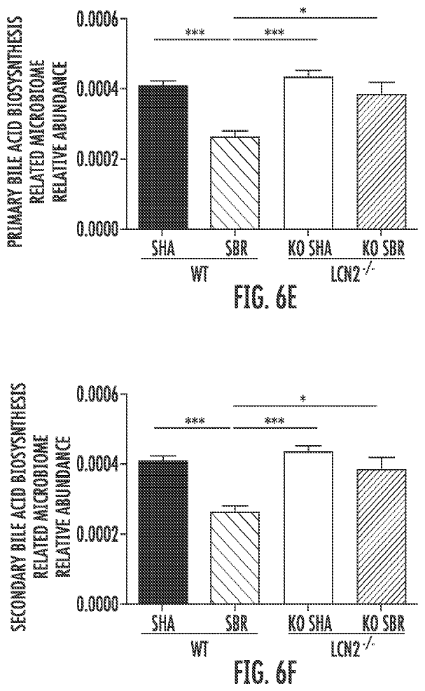

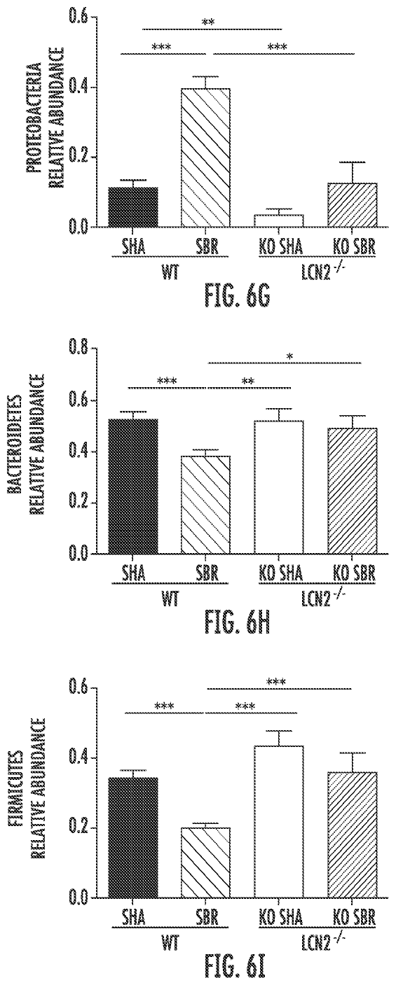

Chao 1 richness (FIG. 6A), Faith' diversity (FIG. 6B), and total OTUs (FIG. 6C) showed that taxa richness and evenness were significant different between WT and LCN2^{-/-} mice. There was significant decrease of alpha diversities in the LCN2^{-/-} mice as compared to the WT mice before and after 75% SBR at POD7. The principal component analysis (PCA) results showed that intestinal microbiome profile was clearly divided into four groups (FIG. 6D). LCN2^{-/-} SBR mice had significantly less pro-inflammatory Proteobacteria (FIG. 6G), more Bacteroidetes (FIG. 6H) and Firmicutes (FIG. 6I) than WT SBR mice. We also observed significant loss of KEGG pathways at level 3 for gut microbiome associated with primary and secondary bile acid biosynthesis in WT mice after 75% SBR, but not in LCN2^{-/-} SBR mice (FIG. 6E-6F). These data indicated that the presence of LCN2 reduced intestinal function during adaptation and led to a more profound dysbiosis.

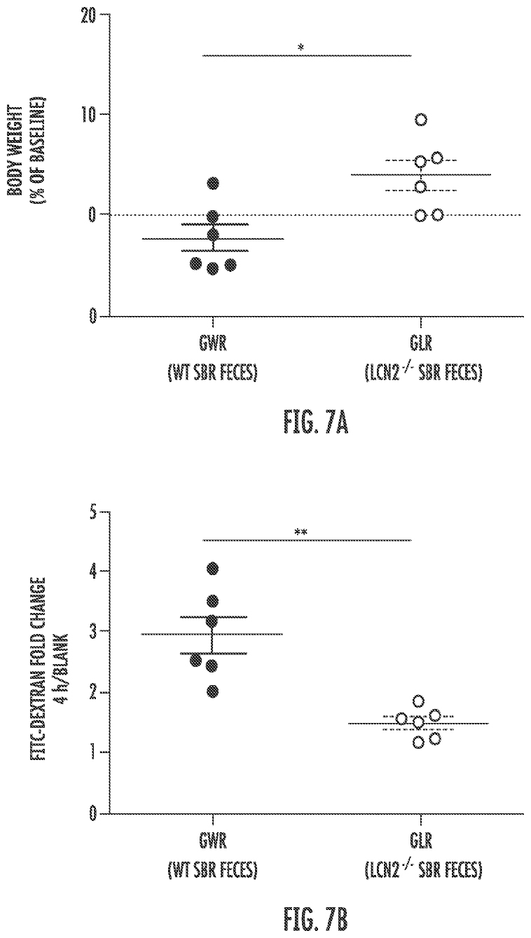

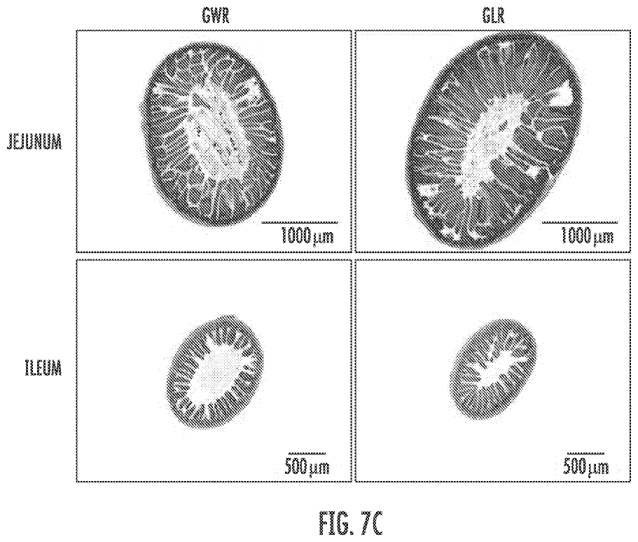

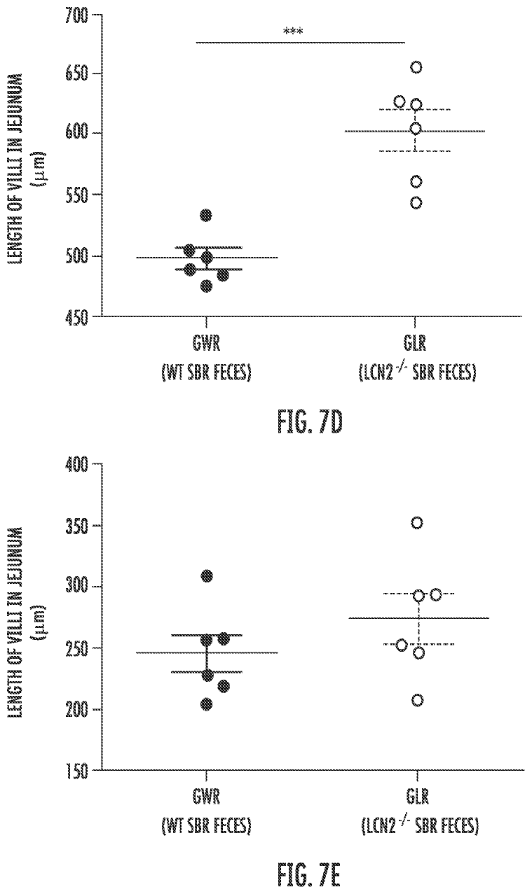

The Pro-inflammatory and Anti-Adaptive Effects of LCN2 can be transferred to Germ-Free Mice via a Fecal Transplant. In order to define the role of the microbiome in our SBS model, we created 4% stool slurry from WT SBR and LCN2^{-/-} SBR mice respectively and performed fecal microbiota transplant in GF mice. GF mice transplanted with WT SBR cecal contents lost 3% of their body weight over 7 days; whereas, GF mice transplanted with LCN2^{-/-} SBR cecal contents gained 6% of their body weight (FIG. 7A). Consistent with the weight loss, intestinal permeability was higher in GF mice transplanted with WT SBR fecal matter as compared to those transplanted with LCN2^{-/-} SBR fecal matter (FIG. 7B). In addition, the jejunal villi were significantly longer in GF mice transplanted with LCN2^{-/-} SBR fecal matter as compared to those transplanted with WT SBR fecal matter (FIG. 7C-7E).

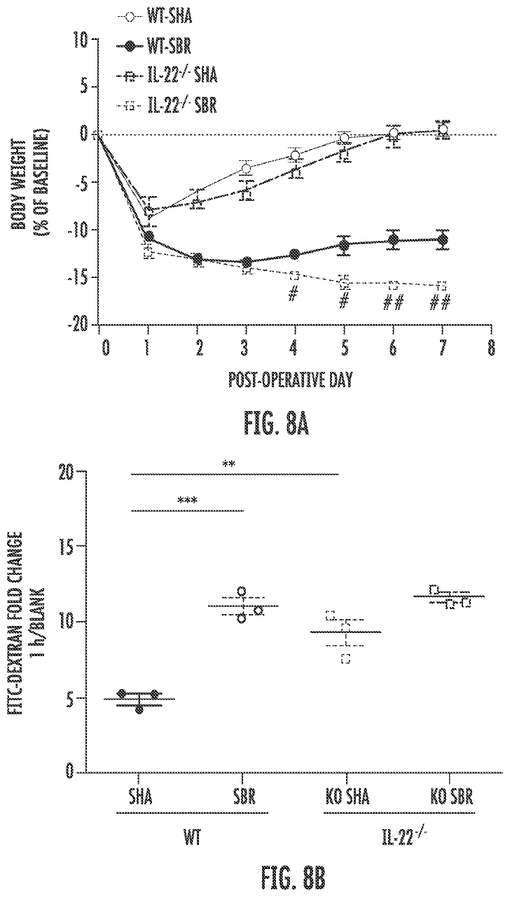

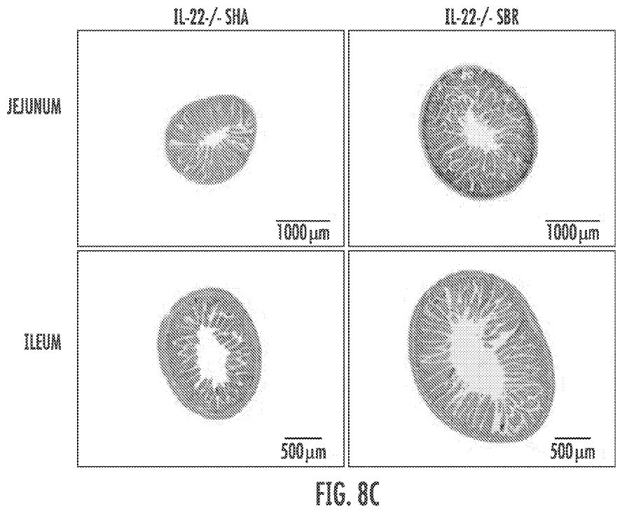

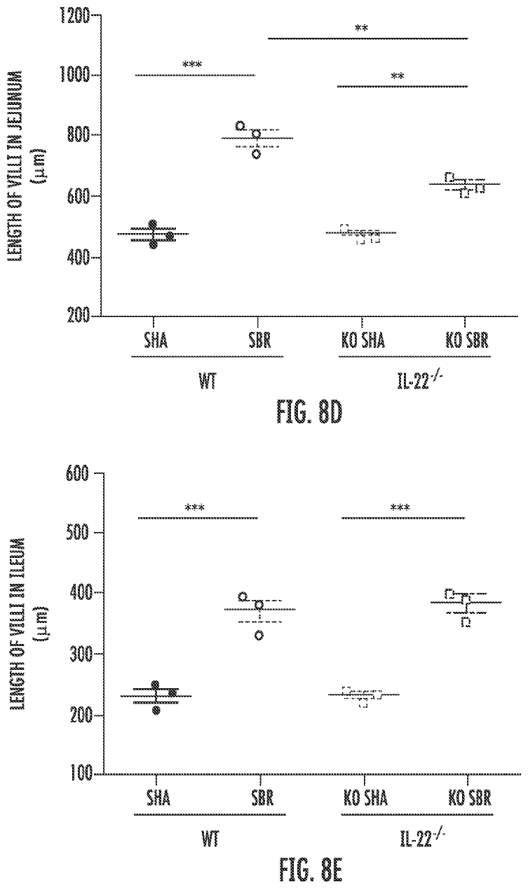

IL-22 Augmented Intestinal Adaptation Following SBR. IL-22 is known to be involved in intestinal barrier homeostasis, and IL-22^{-/-} mice were shown to have had compromised intestinal barrier function as compared to WT mice.²⁴ We hypothesized that the absence of IL-22 would be detrimental to intestinal adaptation following SBS. To test this hypothesis, we subjected IL-22^{-/-} mice to our 75% SBR model. Indeed, we found that IL-22^{-/-} SBR mice lost more body weight and had shorter villi in jejunum than WT SBR mice (FIG. 8A, 8C-8E). Of note, sham-operated IL-22^{-/-} mice had increased intestinal permeability (FIG. 8B).

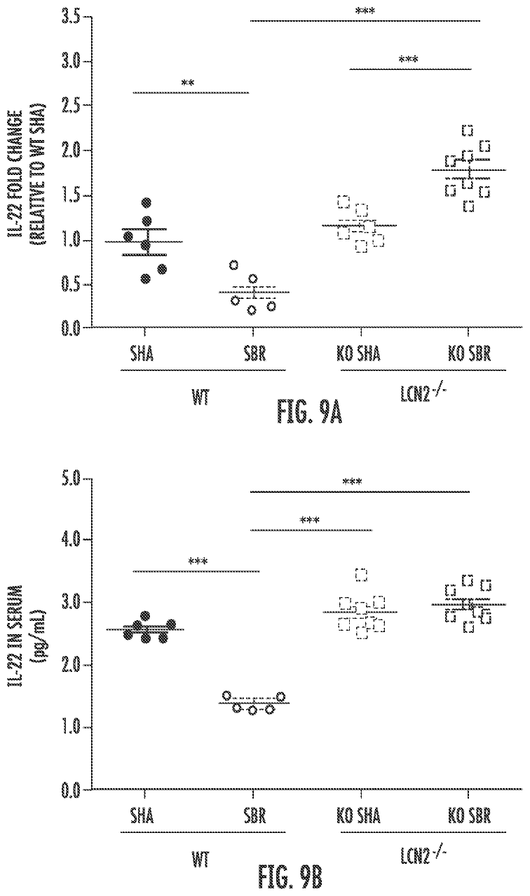

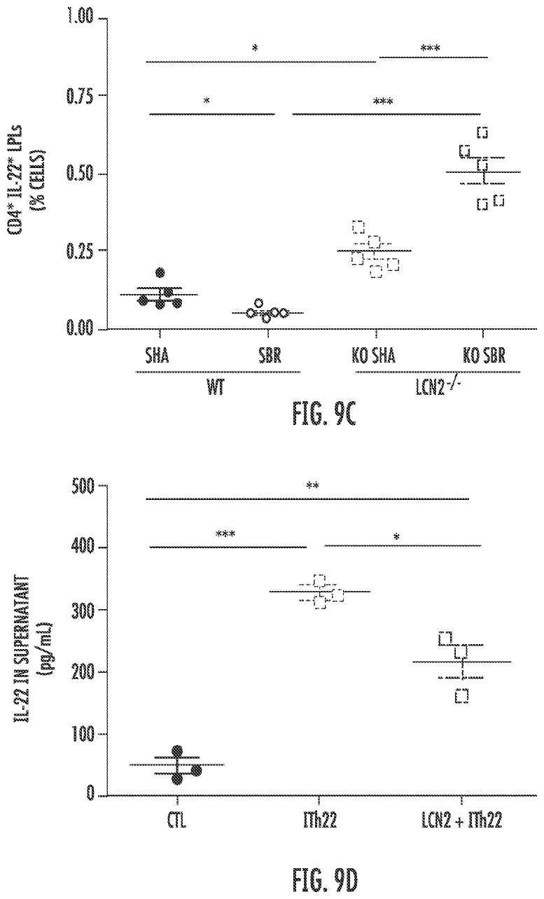

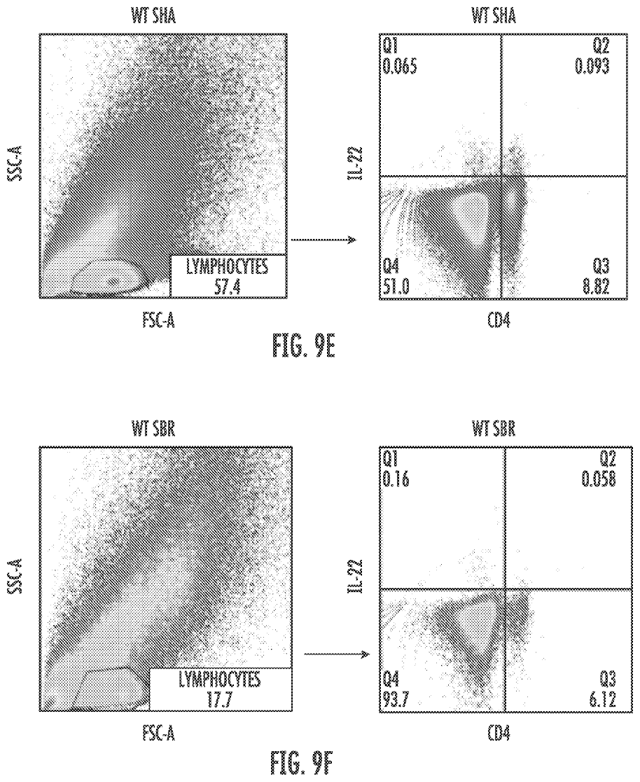

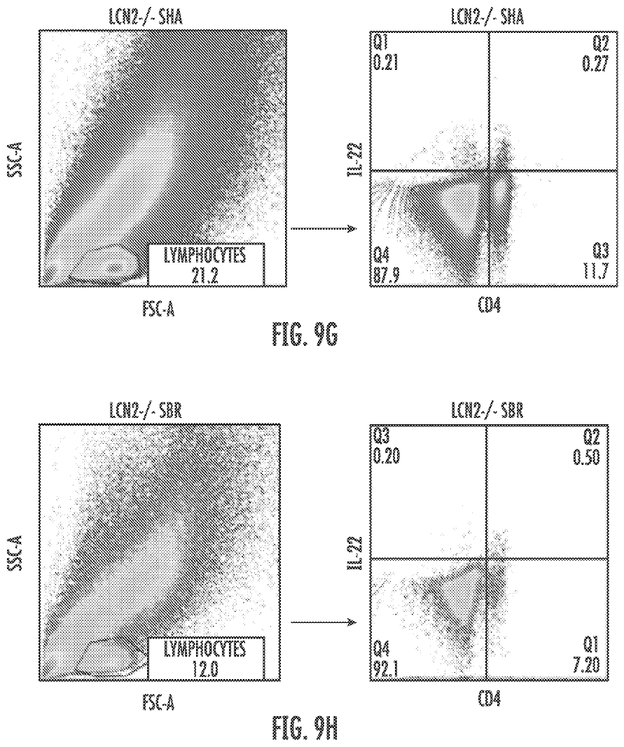

LCN2 Reduced Intestinal Adaptation by Inhibiting IL-22 Expression following 75% SBR. Understanding the significance of IL-22 in SBS, we quantified the gene and protein expression in our WT and LCN2^{-/-} mice. In vivo, comparing to WT SHA mice, transcriptional levels of IL-22 gene in small intestine and IL-22 levels in the serum significantly decreased in WT SBR mice. However, comparing to LCN2^{-/-} SHA mice, IL-22 gene significantly increased in small intestine and IL-22 in the serum didn't decrease in LCN2^{-/-} SBR mice (FIG. 9A-9B). Therefore, we hypothesized that LCN2 may inhibit IL-22 expression and reduce adaptation in SBS. Subsequent isolation of laminal propria lymphocytes (LPLs) from the small intestine were then analyzed by flow cytometry. The percentage of CD4+IL-22+ cells among LPLs significantly decreased following SBR in WT mice while the percentage of these cells increased in SBR LCN2^{-/-} mice (FIG. 9C). In vitro, we successfully polarized naïve T cells to Th22 cells, demonstrated by a significant increase of IL-22 in the supernatant. While the presence of exogenous LCN2 significantly decreased the levels of IL-22 protein expression from Th22 cells (FIG. 9D). Taken together, these data outline a mechanism through which LCN2 reduced intestinal function during adaptation following SBS by inhibiting IL-22 expression.

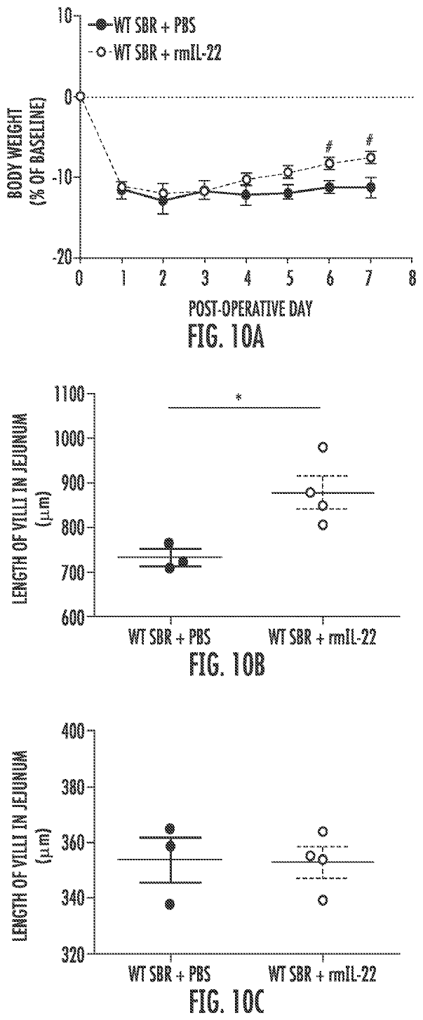

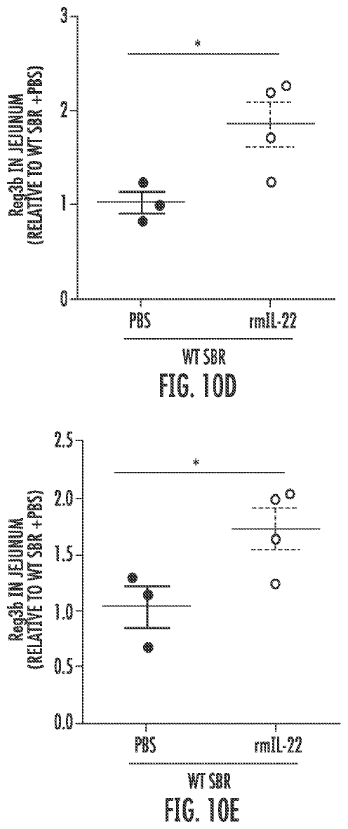

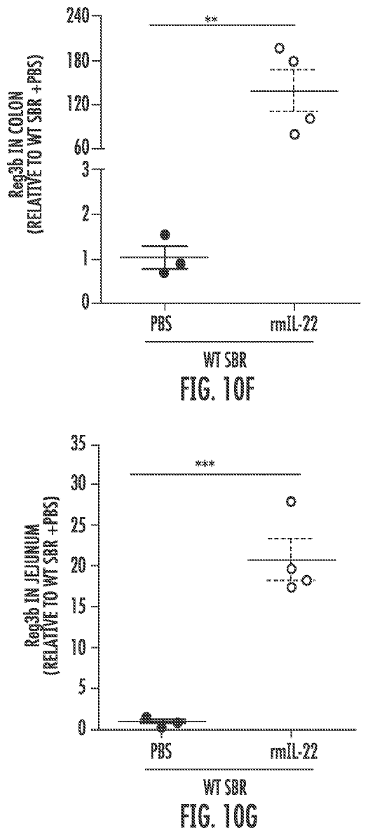

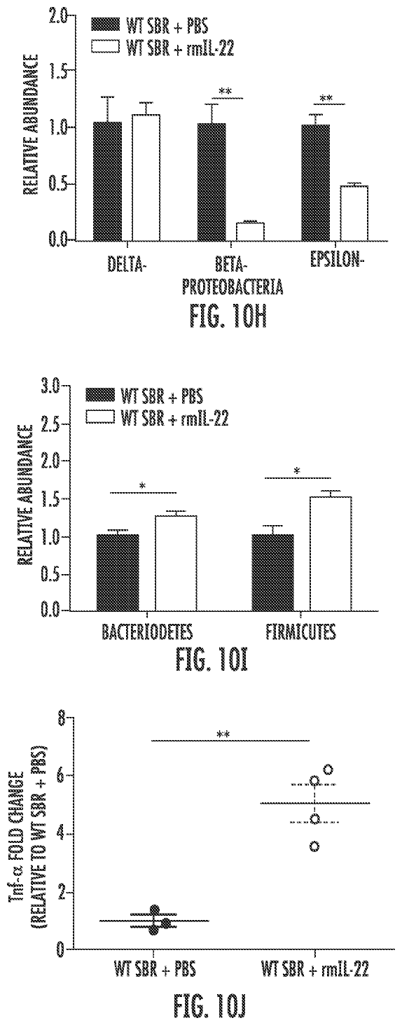

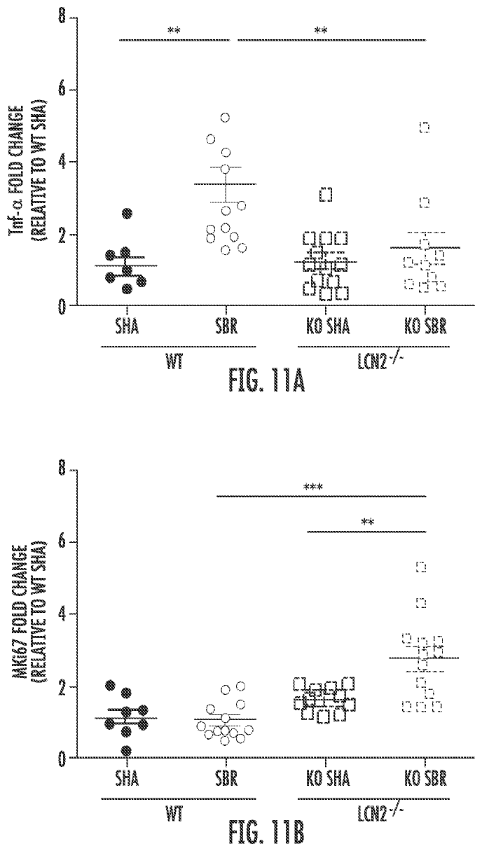

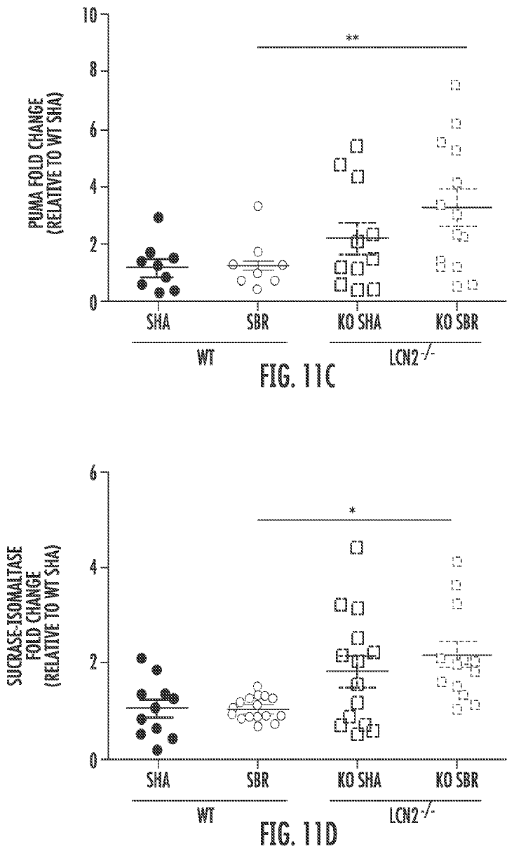

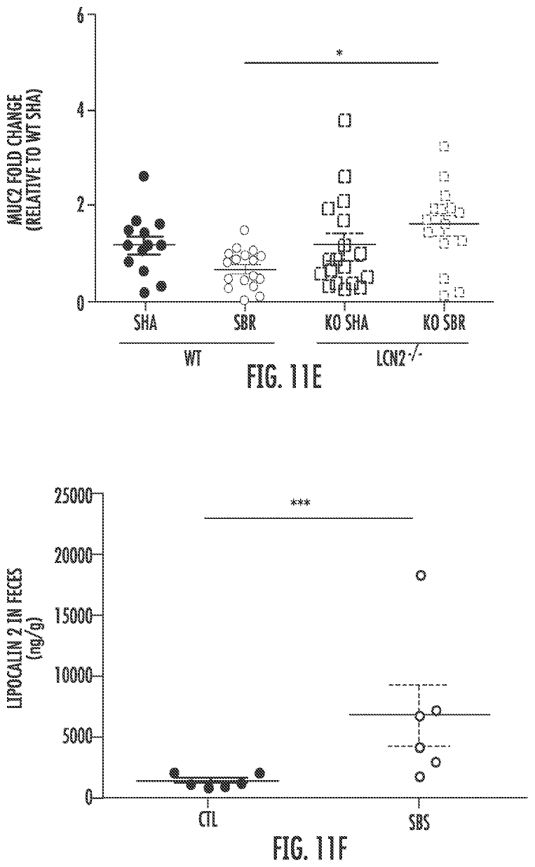

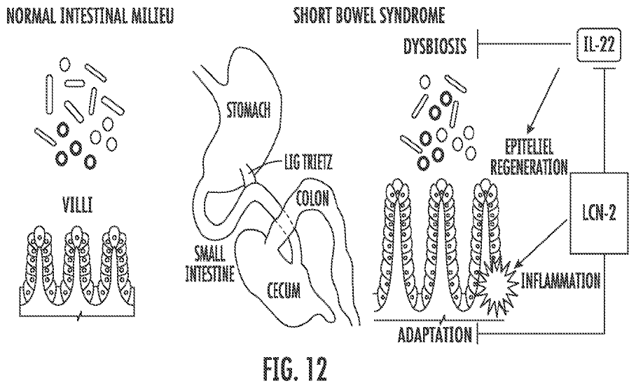

IL-22 Improved Intestinal Adaptation and Counteracted Dysbiosis Following 75% SBR. To determine if restoration of IL-22 could rescue the WT SBR mice from impaired adaptation and dysbiosis, we treated WT SBR mice with either PBS or exogenous rmIL-22 through intraperitoneal injections. At POD 7, PBS-treated WT SBR mice had lost 110% of their body weight; whereas, the rmIL-22-treated WT SBR mice had only lost 7.5% of their body weight (FIG. 10A). Comparing to the PBS-treated group, the rmIL-22-treated mice had significantly longer villi in jejunum (FIG. 10B). While there was no difference in the length of villi in ileum between these two groups (FIG. 10C). We also assessed IL-22 mediated anti-bacterial peptide Reg3b and Reg3g in both jejunum and colon. The rmIL-22 treatment triggered robust increase of Reg3b and Reg3g, especially in the colon (FIG. 10D-10G).

Furthermore, rmIL-22 treatment counteracted intestinal dysbiosis in SBS which was evidenced by decreased Proteobacteria as well as increased Bacteroidetes and Firmicutes (FIG. 10H-10I). However, IL-22 induced inflammation as increased transcriptional levels of Tnf-α in the jejunum (FIG. 10J).

### Discussion

Intestinal adaptation is an important compensatory process after massive bowel resection. In our mouse model of SBS, we had shown that epithelial proliferation, structurally longer villi of residual small intestine, increased intestinal permeability, and intestinal dysbiosis. Intestinal adaptation after 75% SBR in mice was similar to what was seen in human SBS patients, which lent validity to the current mouse model of SBS.¹ It was reported that depletion of gut microbiome altered genes related to cell cycle, lipid biosynthesis, inflammatory response, and antimicrobial factors.²⁵ We depleted gut microbiome with antibiotics cocktail and performed 75% SBR in WT mice. We found that gut microbiome depletion significantly decreased survival, reduced intestinal adaptation and LCN2 expression, which demonstrated the adverse effect from antibiotics and beneficial effects of gut microbiome in SBS. Further investigation will be needed to explore novel therapeutic targets to reduce intestinal dysbiosis and improve intestinal adaptation.

Consistent with our earlier observation that Lcn2 mRNA level increased following common bile duct ligation and Teitelbaum's finding of increased Lcn2 gene expression following 70% SBR in a mouse, we also shown that Lcn2 gene and LCN2 protein expression significantly increased in our 75% SBR mouse model. It was reported that LCN2 could trigger inflammation and play an active role in non-alcoholic steatohepatitis,²⁶ cardiovascular diseases,^{27, 28} and obesity/metabolic syndrome.²⁹ Recent studies had shown that LCN2 significantly increased in patients with inflammatory bowel disease (IBD). Serum and fecal LCN2 had been reported as a marker for the severity of disease in patients.^{30, 31} In our current studies, significant inflammation and detrimental effect of intestinal adaptation were observed in WT mice with increased LCN2 expression following SBR. While LCN2^{-/-} mice which underwent SBR had decreased inflammation and evidences of increased intestinal adaptation with improved weight gain, increased jejunal villi length, increased carbohydrate enzyme expression as compared to WT SBR mice. Given the role of LCN2 in pro-inflammatory conditions, neutralization or inhibition of LCN2 may be a potential therapeutic target to down-regulate intestinal inflammation and improve intestinal adaptation in SBS.

Besides inflammation, LCN2 was identified as bacteriostatic agent in 2002.¹⁴ We originally had hypothesized that increase of LCN2 in SBS was a host defense mechanism, which would prevent dysbiosis and improve intestinal adaptation. Surprisingly, we found that the absence of LCN2 in SBS demonstrated less dysbiosis and better intestinal adaptation. To better understand the role of LCN2 in microbiome after SBR, we performed 16S rRNA sequencing from the cecal content in WT and LCN2^{-/-} mice. We found that the absence of LCN2 resulted in significant changes in diversity and microbial community. Comparing to WT mice, LCN2^{-/-} mice had relative decrease of Proteobacteria as well as increase of Bacteroidetes, Firmicutes, and significant KEGG pathways at level 3 for gut microbiome of primary and secondary bile acid biosynthesis. LCN2 didn't prevent intestinal dysbiosis and improve adaptation as a bacteriostatic agent in SBS.

Host-gut microbiota crosstalk plays an important role in intestinal adaptation.¹¹ GF mice are valuable experimental tools for revealing host-microbe interactions.³² In order to investigate whether the microbiome changes in the absence of LCN2 in SBS would improve intestinal adaptation, we performed fecal transplantation in GF mice. We demonstrated that GF mice with fecal transplantation from LCN2^{-/-} SBR donors had lower intestinal permeability and more weight gain; whereas, GF mice with fecal matters from WT SBR mice donors led to increased intestinal permeability and weight loss. These findings supported that microbiome from LCN2^{-/-} SBR donors had beneficial effects on intestinal adaptation. Fecal microbiota transplantation is best known as the new standard treatment for recurrent Clostridium difficile colitis, inflammatory bowel disease, and obesity/metabolic syndrome.³³ Given the role of LCN2 in microbial diversity and community changes in SBS, inhibition of LCN2 or fecal microbiota transplantation may be potential therapeutic targets to alleviate intestinal dysbiosis and improve intestinal adaptation in SBS.

Since our current studies showed that LCN2 didn't play an important role as anti-bacterial peptide in SBS. We next sought to determine the mechanism behind worse intestinal adaptation in the presence of LCN2. We found that serum IL-22 decreased significantly in WT SBR mice, but not in LCN2^{-/-} SBR mice. Therefore, we hypothesized that LCN2 may inhibit IL-22 in SBS. In vivo, we found decrease of IL-22 gene transcription and CD4+IL-22+ LPLs in the small intestine in WT SBR mice compared to LCN2^{-/-} SBR mice. In vitro, IL-22 expression significantly decreased in Th22 cells in the presence of LCN2. It was reported that IL-22 was able to induce epithelial regeneration by promoting stem cells proliferation.³⁴ IL-22 was required for induction of antibacterial peptides Reg3b and Reg3g and played a key role in host defense against bacterial.²⁴ Choudhry and colleagues found that IL-22 administration restored intestinal barrier integrity and reduced overgrowth of Enterobacteriaceae in the small intestine following ethanol and burn injury in a mouse mode.³⁵ We subjected IL-22^{-/-} mice to 75% SBR and found increased weight loss and intestinal permeability as compared to WT SRB mice. Restoration of IL-22 with exogenous rmIL-22 in our SBS model did improve adaptation and prevent dysbiosis, as evidenced by weight gain, increased villi length, upregulation of Reg3b/Reg3g, reduction of Proteobacteria, and increase of Bacteroidetes and Firmicutes in small intestine. However, besides tissue regeneration, IL-22 has a dual nature, aggravating disease severity by upregulation of inflammation such as inflammatory bowel disease.^{36, 37} The dual nature of IL-22 likely depends on the inflammatory context, which includes the duration and amount of IL-22 present, the overall cytokine milieu, and the tissues involved.³⁸ Therefore, it will be challenging to optimize the treatment dose of IL-22, which maximizes the beneficial effects of tissue regeneration and host-defense as well as minimizes the inflammation induced injury. Restoration of IL-22 by neutralization of LCN2 may be a better option.

In summary, we observed an increase of LCN2 expression following 75% SBR in a mouse model of SBS. This increased LCN2 expression was associated with increased inflammation, decreased intestinal adaptation, increased intestinal permeability and a more profound intestinal dysbiosis following 75% SBR compared to SBR mice without LCN2. Moreover, increased LCN2 expression was associated with decreased IL-22 expression in the serum and CD4+IL-22+ LPLs in the small intestine of WT SBR mice in vivo as well as decreased IL-22 expression through Th22 cells in the presence of LCN2 in vitro, which supported the mechanism that LCN2 reduced intestinal adaptation through inhibition of IL-22 in SBS. Nevertheless, inhibition of LCN2 has the potential to be a prime therapeutic target as exemplified in our rescue experiment. Further exploration will be needed to investigate LCN2 and IL-22 expression in SBS patients who fail to progress to enteral autonomy. This type of mechanism-focused therapy to augment intestinal adaptation and decrease parenteral nutrition dependence and the associated infectious complications in SBS without the use of antibiotics warrants continued investigation.

### REFERENCES

1. Duggan C P, Jaksic T. Pediatric Intestinal Failure. N Engl J Med 2017; 377:666-675.
2. Squires R H, Duggan C, Teitelbaum D H, et al. Natural history of pediatric intestinal failure: initial report from the Pediatric Intestinal Failure Consortium. J Pediatr 2012; 161:723-8 e2.
3. Fullerton B S, Hong C R, Jaksic T. Long-term outcomes of pediatric intestinal failure. Semin Pediatr Surg 2017; 26:328-335.
4. Davidovics Z H, Carter B A, Luna R A, et al. The Fecal Microbiome in Pediatric Patients With Short Bowel Syndrome. JPEN J Parenter Enteral Nutr 2016; 40:1106-1113.
5. Engstrand Lilja H, Wefer H, Nystrom N, et al. Intestinal dysbiosis in children with short bowel syndrome is associated with impaired outcome. Microbiome 2015; 3:18.
6. Piper H G, Fan D, Coughlin L A, et al. Severe Gut Microbiota Dysbiosis Is Associated With Poor Growth in Patients With Short Bowel Syndrome. JPEN J Parenter Enteral Nutr 2017; 41:1202-1212.
7. Sears C L. Molecular physiology and pathophysiology of tight junctions V. assault of the tight junction by enteric pathogens. Am J Physiol Gastrointest Liver Physiol 2000; 279:G1129-34.
8. Kelly D A. Preventing parenteral nutrition liver disease. Early Hum Dev 2010; 86:683-7.
9. Deitch E A, Sittig K, Li M, et al. Obstructive jaundice promotes bacterial translocation from the gut. Am J Surg 1990; 159:79-84.
10. Alaish S M, Smith A D, Timmons J, et al. Gut microbiota, tight junction protein expression, intestinal resistance, bacterial translocation and mortality following cholestasis depend on the genetic background of the host. Gut Microbes 2013; 4:292-305.
11. Marchix J, Goddard G, Helmrath M A. Host-Gut Microbiota Crosstalk in Intestinal Adaptation. Cell Mol Gastroenterol Hepatol 2018; 6:149-162.
12. Nasioudis D, Witkin S S. Neutrophil gelatinase-associated lipocalin and innate immune responses to bacterial infections. Med Microbiol Immunol 2015; 204:471-9.
13. Flo T H, Smith K D, Sato S, et al. Lipocalin 2 mediates an innate immune response to bacterial infection by sequestrating iron. Nature 2004; 432:917-21.
14. Goetz D H, Holmes M A, Borregaard N, et al. The neutrophil lipocalin NGAL is a bacteriostatic agent that interferes with siderophore-mediated iron acquisition. Mol Cell 2002; 10:1033-43.
15. Berger T, Togawa A, Duncan G S, et al. Lipocalin 2-deficient mice exhibit increased sensitivity to Escherichia coli infection but not to ischemia-reperfusion injury. Proc Natl Acad Sci USA 2006; 103:1834-9.
16. Alaish S M, Timmons J, Smith A, et al. Candidate Genes for Limiting Cholestatic Intestinal Injury Identified by Gene Expression Profiling. Physiol Rep 2013; 1.
17. Wildhaber B E, Yang H, Coran A G, et al. Gene alteration of intestinal intraepithelial lymphocytes in response to massive small bowel resection. Pediatr Surg Int 2003; 19:310-5.
18. Helmrath M A, VanderKolk W E, Can G, et al. Intestinal adaptation following massive small bowel resection in the mouse. J Am Coll Surg 1996; 183:441-9.
19. Daquigan N, Seekatz A M, Greathouse K L, et al. High-resolution profiling of the gut microbiome reveals the extent of Clostridium difficile burden. NPJ Biofilms Microbiomes 2017; 3:35.
20. Drewes J L, White J R, Dejea C M, et al. High-resolution bacterial 16S rRNA gene profile meta-analysis and biofilm status reveal common colorectal cancer consortia. NPJ Biofilms Microbiomes 2017; 3:34.
21. Grim C J, Daquigan N, Lusk Pfefer T S, et al. High-Resolution Microbiome Profiling for Detection and Tracking of Salmonella enterica. Front Microbiol 2017; 8:1587.
22. Bedoya S K, Wilson T D, Collins E L, et al. Isolation and th17 differentiation of naive CD4 T lymphocytes. J Vis Exp 2013:e50765.
23. Plank M W, Kaiko G E, Maltby S, et al. Th22 Cells Form a Distinct Th Lineage from Th17 Cells In Vitro with Unique Transcriptional Properties and Tbet-Dependent Th1 Plasticity. J Immunol 2017; 198:2182-2190.
24. Zheng Y, Valdez P A, Danilenko D M, et al. Interleukin-22 mediates early host defense against attaching and effacing bacterial pathogens. Nat Med 2008; 14:282-9.
25. Reikvam D H, Erofeev A, Sandvik A, et al. Depletion of murine intestinal microbiota: effects on gut mucosa and epithelial gene expression. PLoS One 2011; 6:e17996.
26. Ye D, Yang K, Zang S, et al. Lipocalin-2 mediates non-alcoholic steatohepatitis by promoting neutrophil-macrophage crosstalk via the induction of CXCR2. J Hepatol 2016; 65:988-997.
27. Tarin C, Fernandez-Garcia C E, Burillo E, et al. Lipocalin-2 deficiency or blockade protects against aortic abdominal aneurysm development in mice. Cardiovasc Res 2016; 111:262-73.
28. Wu G, Li H, Fang Q, et al. Elevated circulating lipocalin-2 levels independently predict incident cardiovascular events in men in a population-based cohort.

Arterioscler Thromb Vasc Biol 2014; 34:2457-64.
29. Law I K, Xu A, Lam K S, et al. Lipocalin-2 deficiency attenuates insulin resistance associated with aging and obesity. Diabetes 2010; 59:872-82.
30. Oikonomou K A, Kapsoritakis A N, Theodoridou C, et al. Neutrophil gelatinase-associated lipocalin (NGAL) in inflammatory bowel disease: association with pathophysiology of inflammation, established markers, and disease activity. J Gastroenterol 2012; 47:519-30.
31. Thorsvik S, Damas J K, Granlund A V, et al. Fecal neutrophil gelatinase-associated lipocalin as a biomarker for inflammatory bowel disease. J Gastroenterol Hepatol 2017; 32:128-135.
32. Al-Asmakh M, Zadjali F. Use of Germ-Free Animal Models in Microbiota-Related Research. J Microbiol Biotechnol 2015; 25:1583-8.
33. Staley C, Khoruts A, Sadowsky M J. Contemporary Applications of Fecal Microbiota Transplantation to Treat Intestinal Diseases in Humans. Arch Med Res 2017; 48:766-773.
34. Lindemans C A, Calafiore M, Mertelsmann A M, et al. Interleukin-22 promotes intestinal-stem-cell-mediated epithelial regeneration. Nature 2015; 528:560-564.
35. Hammer A M, Morris N L, Cannon A R, et al. Interleukin-22 Prevents Microbial Dysbiosis and Promotes Intestinal Barrier Regeneration Following Acute Injury.

Shock 2017; 48:657-665.
36. Sugimoto K, Ogawa A, Mizoguchi E, et al. IL-22 ameliorates intestinal inflammation in a mouse model of ulcerative colitis. J Clin Invest 2008; 118:534-44.
37. Zenewicz L A, Yancopoulos G D, Valenzuela D M, et al. Innate and adaptive interleukin-22 protects mice from inflammatory bowel disease. Immunity 2008; 29:947-57.
38. Alabbas S Y, Begun J, Florin T H, et al. The role of IL-22 in the resolution of sterile and nonsterile inflammation. Clin Transl Immunology 2018; 7:e1017.

### Example 2: Aryl Hydrocarbon Receptor (AHR) is a Target for Children with SBS

Based on our preliminary data, our central hypothesis is that LCN2 and its role in both the innate and adaptive immune responses following massive SBR determine the degree of intestinal inflammation, barrier dysfunction and dysbiosis that limits intestinal adaptation. The aryl hydrocarbon receptor (AHR) serves as a common link between the two immune responses, for its activation stimulates type 3 innate lymphoid cells (ILC3) to secrete IL-22 while also stimulating intraepithelial lymphocytes (IELs) to secrete IL-1013. Thus, we further hypothesize that adaptation can be improved by targeting AHR to attenuate inflammation, increase cell proliferation and strengthen the intestinal barrier.

We will test this hypothesis via three specific aims:

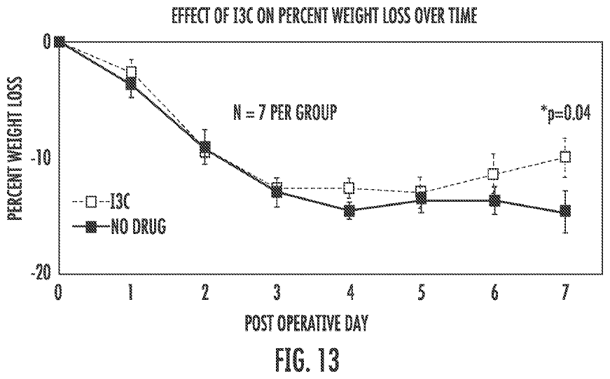

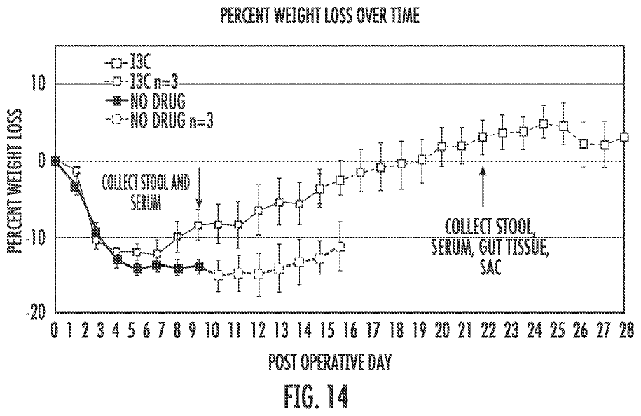

1. Determine the role of LCN2 expression and AHR activation on the function of ILC3 cells in characterizing the microbiome and intestinal adaptation following SBS in a mouse model. Our preliminary data shows increased intestinal AHR expression following SBR. Moreover, we found that I3C supplementation, an AHR agonist, increases weight gain following SBR. See FIGS. 13-14. Our working hypothesis is that enhanced AHR activation can modulate ILC3 cells after massive SBR to decrease intestinal inflammation and improve adaptation. Here, will delineate the effects of AHR activation on ILC3 cells and IL-22 production in our wild-type (WT) and LCN2−/− SBR mice, as well as in AHR−/− and AHR−/− LCN2−/− mice following SBR.

2. Determine the role of LCN2 expression and AHR activation on the function of IELs in characterizing the microbiome and intestinal adaptation following SBS in a mouse model. Preliminary RNA sequencing data, confirmed by PCR, has revealed downregulation in many adaptive immune response genes following SBR. We hypothesize that increased LCN2 expression following SBR inhibits expression of IL-10 by IELs and reduces adaptation; whereas, AHR activation increases expression of IL-10 by ILEs and adaptation. We will activate AHR and study the expression of the candidate genes and the effects on IELs and IL-10 in our WT, LCN2−/−, AHR−/− and AHR−/−LCN2−/− mice following SBR.

3. Determine whether AHR activation and reduced LCN2 expression limits inflammation and improves adaptation in human SBS enteroids. Our pilot data shows increased levels of pro-inflammatory LCN2 in the stool of SBS children compared to healthy controls. We hypothesize that AHR activation and reduced inflammation will correlate with improved adaptation in SBS children. Prepared from intestinal biopsies from SBS children, human enteroids will be exposed to I3C, rhIL-22, LCN2Ab or PBS and stained for proliferation, apoptosis and AHR and LCN2 expression.

Aryl hydrocarbon receptor (AHR) is an essential regulator of gut barrier protection and mucosal immunity, as evidenced by the phenotype of AHR-deficient mice, which are more susceptible to epithelial damage, dysbiosis and colitis with reduced ILC3 cells. Importantly, AHR is down-regulated in intestinal tissue of patients with inflammatory bowel disease. Recently, AHR agonists have been shown to increase IL-22 production by ILC3 cells and CD4+ T cells and accelerate mucosal healing in a murine model of colitis. Hence, we hypothesize that enhanced AHR activation can modulate ILC3 cells after massive SBR to decrease intestinal inflammation and improve adaptation.

In one experiment, AHR agonists are studied in our SBR model. The purpose of this experiment is to study AHR activation via a dietary supplement, which has tremendous potential as a translational approach. Under our ACUC-approved protocol, we will perform a 75% SBR on the same four groups [n=12 mice in each group] of C57Bl/6J mice: WT, LCN2−/−, AHR−/− and AHR−/−LCN2−/−. Sham-operated WT, LCN2−/−, AHR−/− and AHR−/−LCN2−/− C57Bl/6J mice [n=12 mice in each group] will serve as controls. Mice will be randomized to receive either the AHR agonist, indole-3-carbinol (I3C) (Sigma, 17256, St Louis, Mo.) at a dose of 15-20 mg/kg, dissolved in DMSO, or DMSO alone mixed into the liquid diet. The rodent liquid diet containing I3C will be maintained for the duration of the postoperative period. Control mice will receive DMSO only. Mice will be weighed daily. One week later, the mice will be anesthetized for tissue collection and analyses performed.

1. A method for treating a patient having Short Bowel Syndrome (SBS) who is undergoing parenteral nutrition comprising the steps of:
(a) measuring lipocalin-2 (LCN2) in a sample obtained from the patient; and
(b) reducing or eliminating parenteral nutrition if the measured level of LCN2 is below a control level or treating the patient with IL-22, a LCN2 inhibitor and/or an AHR agonist if the measured level of LCN2 is above the control level.

2. The method of claim 1, wherein the sample is a stool sample.

3. The method of claim 1, wherein the sample is a blood sample.

4. The method of claim 1, wherein step (a) is accomplished using a polymerase chain reaction (PCR) assay.

5. The method of claim 4, wherein the PCR assay uses a primer comprising SEQ ID NO:3 and/or SEQ ID NO:4.

6. The method of claim 1, wherein step (a) is accomplished using an immunoassay.

7. The method of claim 1, wherein the patient undergoes a fecal transplant if the measured level of LCN2 is above the control level.

8. The method of claim 1, wherein the LCN2 inhibitor is a small molecule, an antibody or an inhibitory nucleic acid molecule.

9. The method of claim 8, wherein the inhibitory nucleic acid molecule is an siRNA, shRNA, antisense RNA or a ribozyme.

10. A method comprising performing a PCR assay to measure LCN2 in a sample obtained from a patient having SBS.

11. The method of claim 8, wherein the PCR assay uses a primer comprising SEQ ID NO:3 and/or SEQ ID NO:4.

12. A method for treating intestinal dysbiosis associated with SBS in a patient comprising the step of administering to the patient IL-22, a LCN2 inhibitor and/or an AHR agonist.

13. The method of claim 12, wherein the LCN2 inhibitor is a small molecule, an antibody or an inhibitory nucleic acid molecule.

14. The method of claim 13, wherein the inhibitory nucleic acid molecule is an siRNA, shRNA, antisense RNA or a ribozyme.

15. A method for assessing intestinal adaptation of a patient having SBS comprising the steps of:
(a) measuring LCN2 in a sample obtained from the patient;
(b) comparing the measured LCN2 of step (a) to a control level, wherein LCN2 expression below the control level correlates with intestinal adaptation, and wherein LCN2 expression above the control level does not correlate with intestinal adaptation.

16. The method of claim 15, wherein the patient whose LCN2 expression correlates with intestinal adaptation reduces or eliminates parenteral nutrition.

17. The method of claim 15, wherein the patient whose LCN2 expression does not correlate with intestinal adaptation undergoes one or more of IL-22 treatment, LCN2 inhibitor treatment, AHR agonist treatment and a fecal transplant.

18. The method of claim 17, wherein the LCN2 inhibitor is a small molecule, an antibody or an inhibitory nucleic acid molecule.

19. The method of claim 18, wherein the inhibitory nucleic acid molecule is an siRNA, shRNA, antisense RNA or a ribozyme.

20. The method of claim 15, wherein the sample is a blood or stool sample.

21. (canceled)
